SEQUENCE LISTING (85 sequences)

SEQ ID NO: 1 - DNA, 18 bp, artificial sequence
  misc_feature (1)..(18): primer
tgctatgcgcgagctgcg

SEQ ID NO: 2 - DNA, 24 bp, artificial sequence
  misc_feature (1)..(24): primer
aataaagtgacagatagctgggca

SEQ ID NO: 3 - DNA, 19 bp, artificial sequence
  misc_feature (1)..(19): PRIMER
ccttcataaccttcgcccg

SEQ ID NO: 4 - DNA, 43 bp, artificial sequence
  misc_feature (1)..(43): primer
tttttacgaaggactctaacgtttaacatcctttgccattttt

SEQ ID NO: 5 - DNA, 31 bp, artificial sequence
  misc_feature (1)..(31): probe
acgaaggactctaacgtttaacatcctttgc

SEQ ID NO: 6 - DNA, 31 bp, artificial sequence
  misc_feature (1)..(31): probe
gcaaaggatgttaaacgttagagtccttcgt

SEQ ID NO: 7 - DNA, 20 bp, artificial sequence
  misc_feature (1)..(20): primer
cgcacaatcccactatcctt

SEQ ID NO: 8 - DNA, 20 bp, artificial sequence
  misc_feature (1)..(20): primer
gcctcccagaagtagacgtc

SEQ ID NO: 9 - DNA, 40 bp, artificial sequence
  misc_feature (1)..(40): primer
tttttaagaaacccttactctagcgaagatcctctttttt

SEQ ID NO: 10 - DNA, 28 bp, artificial sequence
  misc_feature (1)..(28): probe
aagaaacccttactctagcgaagatcct

SEQ ID NO: 11 - DNA, 28 bp, artificial sequence
  misc_feature (1)..(28): probe
aggatcttcgctagagtaagggtttctt

SEQ ID NO: 12 - DNA, 21 bp, artificial sequence
  misc_feature (1)..(21): primer
cccatcgacatcagcctgagc

SEQ ID NO: 13 - DNA, 20 bp, artificial sequence
  misc_feature (1)..(20): primer
atctcgcttccgtgcttagc

SEQ ID NO: 14 - DNA, 20 bp, artificial sequence
  misc_feature (1)..(20): primer
caggaaggcgtcccactggc

SEQ ID NO: 15 - DNA, 20 bp, artificial sequence
  misc_feature (1)..(20): primer
ggtcaggctcaggctgatgt

SEQ ID NO: 16 - DNA, 31 bp, artificial sequence
  misc_feature (1)..(31): probe
tttttatgtccaccaggcccagcacgttttt

SEQ ID NO: 17 - DNA, 21 bp, artificial sequence
  misc_feature (1)..(21): probe
tccaccaggcccagcacgaag

SEQ ID NO: 18 - DNA, 19 bp, artificial sequence
  misc_feature (1)..(19): probe
aggcccagcacgaagccgg

SEQ ID NO: 19 - DNA, 23 bp, artificial sequence
  misc_feature (1)..(23): probe
tgagcaaccccgaggtggaggtg

SEQ ID NO: 20 - DNA, 25 bp, artificial sequence
  misc_feature (1)..(25): probe
ccggcttcgtgctgggcctggtgga

SEQ ID NO: 21 - DNA, 23 bp, artificial sequence
  misc_feature (1)..(23): probe
cacctccacctcggggttgctca

SEQ ID NO: 22 - DNA, 19 bp, artificial sequence
  misc_feature (1)..(19): primer
gctcctacaaatgccatca

SEQ ID NO: 23 - DNA, 25 bp, artificial sequence
  misc_feature (1)..(25): primer
gaagatagtggaaaaggaaggtggc

SEQ ID NO: 24 - DNA, 21 bp, artificial sequence
  misc_feature (1)..(21): primer
ggaaacctcctcggattccat

SEQ ID NO: 25 - DNA, 26 bp, artificial sequence
  misc_feature (1)..(26): primer
cccttacgtcagtggagatatcacat

SEQ ID NO: 26 - DNA, 22 bp, artificial sequence
  misc_feature (1)..(22): primer
cttgctttgaagacgtggttgg

SEQ ID NO: 27 - DNA, 19 bp, artificial sequence
  misc_feature (1)..(19): primer
gatgctcctcgtgggtggg

SEQ ID NO: 28 - DNA, 22 bp, artificial sequence
  misc_feature (1)..(22): probe
gaaaggccatcgttgaagatgc

SEQ ID NO: 29 - DNA, 25 bp, artificial
  misc_feature (1)..(25): probe
tgccgacagtggtcccaaagatgga

SEQ ID NO: 30 - DNA, 23 bp, artificial sequence
  misc_feature (1)..(23): probe
ggcatcttcaacgatggcctttc

SEQ ID NO: 31 - DNA, 25 bp, artificial sequence
  misc_feature (1)..(25): probe
tccatctttgggaccactgtcggca

SEQ ID NO: 32 - DNA, 19 bp, artificial sequence
  misc_feature (1)..(19): primer
tgctcacccagaaacgctg

SEQ ID NO: 33 - DNA, 21 bp, artificial sequence
  misc_feature (1)..(21): primer
ttcttcggggcgaaaactctc

SEQ ID NO: 34 - DNA, 30 bp, artificial sequence
  misc_feature (1)..(30): probe
gtaaaagatgctgaagatcagttgggtgca

SEQ ID NO: 35 - DNA, 30 bp, artificial sequence
  misc_feature (1)..(30): probe
tgcacccaactgatcttcagcatcttttac

SEQ ID NO: 36 - DNA, 20 bp, artificial sequence
  misc_feature (1)..(20): primer
gaatcctgttgccggtcttg

SEQ ID NO: 37 - DNA, 23 bp, artificial sequence
  misc_feature (1)..(23): primer
aatttatcctagtttgcgcgcta

SEQ ID NO: 38 - DNA, 33 bp, artificial sequence
  misc_feature (1)..(33): probe
tttatgagatgggtttttatgattagagtcccg

SEQ ID NO: 39 - DNA, 33 bp, artificial sequence
  misc_feature (1)..(33): probe
cgggactctaatcataaaaacccatctcataaa

SEQ ID NO: 40 - DNA, 100 bp, artificial sequence
  misc_feature (1)..(100): synthetic sequence for PCR control
cgcagcgtttcaagcagcacatcatcgatctaatcgagcagacggtacgatcagacgctgtcatacgcataatcgatacgcgatactgcccgctaactgg

SEQ ID NO: 41 - DNA, 18 bp, artificial sequence
  misc_feature (1)..(18): primer
cgcagcgtttcaagcagc

SEQ ID NO: 42 - DNA, 20 bp, artificial sequence
  misc_feature (1)..(20): primer
ccagttagcgggcagtatcg

SEQ ID NO: 43 - DNA, 18 bp, artificial sequence
  misc_feature (1)..(18): primer
cgcagcgtttcaagcagc

SEQ ID NO: 44 - DNA, 20 bp, artificial sequence
  misc_feature (1)..(20): primer
ccagttagcgggcagtatcg

SEQ ID NO: 45 - DNA, 25 bp, artificial sequence
  misc_feature (1)..(25): probe
agcagacggtacgatcagacgctgt

SEQ ID NO: 46 - DNA, 25 bp, artificial sequence
  misc_feature (1)..(25): probe
acagcgtctgatcgtaccgtctgct

SEQ ID NO: 47 - DNA, 23 bp, artificial sequence
  misc_feature (1)..(23): primer
ttggactagaaatctcgtgctga

SEQ ID NO: 48 - DNA, 22 bp, artificial sequence
  misc_feature (1)..(22): primer
gctacatagggagccttgtcct

SEQ ID NO: 49 - DNA, 33 bp, artificial sequence
  misc_feature (1)..(33): primer
tttttcaatccacacaaacgcacggctattttt

SEQ ID NO: 50 - DNA, 19 bp, artificial sequence
  misc_feature (1)..(19): probe
caatccacacaaacgcacg

SEQ ID NO: 51 - DNA, 19 bp, artificial sequence
  misc_feature (1)..(19): probe
cgtgcgtttgtgtggattg

SEQ ID NO: 52 - DNA, 22 bp, artificial sequence
  misc_feature (1)..(22): primer
ccagttaggccagttacccaga

SEQ ID NO: 53 - DNA, 30 bp, artificial sequence
  misc_feature (1)..(30): primer
tgggaactactcacacattattatagagag

SEQ ID NO: 54 - DNA, 24 bp, artificial sequence
  misc_feature (1)..(24): probe
agactggtgatttcagcgggcatg

SEQ ID NO: 55 - DNA, 21 bp, artificial sequence
  misc_feature (1)..(21): probe
aaggatccggtgcatggccgg

SEQ ID NO: 56 - DNA, 22 bp, artificial sequence
  misc_feature (1)..(22): probe capture
gccggccatgcaccggatcctt

SEQ ID NO: 57 - DNA, 23 bp, artificial sequence
  misc_feature (1)..(23): primer
gtcatttcaggaccaggattcac

SEQ ID NO: 58 - DNA, 23 bp, artificial sequence
  misc_feature (1)..(23): primer
cctctattctggatgttgttgcc

SEQ ID NO: 59 - DNA, 28 bp, artificial sequence
  misc_feature (1)..(28): probe
gaagaattcagcctaaccaagtcgcctc

SEQ ID NO: 60 - DNA, 23 bp, artificial sequence
  misc_feature (1)..(23): primer
ggtcagatcgtgagcttctacca

SEQ ID NO: 61 - DNA, 20 bp, artificial sequence
  misc_feature (1)..(20): primer
cgcatgaaagcttcccagat

SEQ ID NO: 62 - DNA, 24 bp, artificial sequence
  misc_feature (1)..(24): probe
gctgaacaccctgtggccagtgaa

SEQ ID NO: 63 - DNA, 21 bp, artificial sequence
  misc_feature (1)..(21): primer
gccagttaggccagttaccca

SEQ ID NO: 64 - DNA, 24 bp, artificial sequence
  misc_feature (1)..(24): primer
tgagcgaaaccctataagaaccct

SEQ ID NO: 65 - DNA, 77 bp, artificial sequence
  misc_feature (1)..(77): synthetic sequence
gtcatttcaggaccaggattcactggaggcgacttggttaggctgaattcttccggcaacaacatccagaatagagg

SEQ ID NO: 66 - DNA, 83 bp, artificial sequence
  misc_feature (1)..(83): synthetic template
ggtcagatcgtgagcttctaccagttcctgctgaacaccctgtggccagtgaacgacaccgccatctgggaagctttcatgcg

SEQ ID NO: 67 - DNA, 25 bp, artificial sequence
  misc_feature (1)..(25): primer
attgatgtgatatctccactgacgt

SEQ ID NO: 68 - DNA, 26 bp, artificial sequence
  misc_feature (1)..(26): primer
actaagggtttcttatatgctcaaca

SEQ ID NO: 69 - DNA, 20 bp, artificial sequence
  misc_feature (1)..(20): primer
cttggcggcttatctgtctc

SEQ ID NO: 70 - DNA, 20 bp, artificial sequence
  misc_feature (1)..(20): primer
gctgctgtagctggcctaat

SEQ ID NO: 71 - DNA, 25 bp, artificial sequence
  misc_feature (1)..(25): probe
tcgacatgtctccggagaggagacc

SEQ ID NO: 72 - DNA, 23 bp, artificial sequence
  misc_feature (1)..(23): probe
ctgagcaaccccgaggtggaggt

SEQ ID NO: 73 - DNA, 25 bp, artificial sequence
  misc_feature (1)..(25): probe
gcatgcccgctgaaatcaccagtct

SEQ ID NO: 74 - DNA, 29 bp, artificial sequence
  misc_feature (1)..(29): probe
ggaggcgacttggttaggctgaattcttc

SEQ ID NO: 75 - DNA, 24 bp, artificial sequence
  misc_feature (1)..(24): probe
tgctgaacaccctgtggccagtga

SEQ ID NO: 76 - DNA, 22 bp, artificial
  misc_feature (1)..(22): primer
tgttaggcgtcatcatctgtgg

SEQ ID NO: 77 - DNA, 21 bp, artificial sequence
  misc_feature (1)..(21): primer
tgcagcaactgttggccttac

SEQ ID NO: 78 - DNA, 26 bp, artificial sequence
  misc_feature (1)..(26): probe
atcatcactggcatcgtctgaagcgg

SEQ ID NO: 79 - DNA, 41 bp, artificial sequence
  misc_feature (1)..(41): primer
gcaggcugcucaugucuguaggaacuuggcaucauauuacc

SEQ ID NO: 80 - DNA, 38 bp, artificial sequence
  misc_feature (1)..(38): primer
gcaggcugcucaugucuguuggacgagcuucuacuggc

SEQ ID NO: 81 - DNA, 18 bp, artificial sequence
  misc_feature (1)..(18): primer
gcaggctgctcatgtctg

SEQ ID NO: 82 - DNA, 41 bp, artificial sequence
  misc_feature (1)..(41): primer
gcaggctgctcatgtctguaggaacuuggcaucauauuacc

SEQ ID NO: 83 - DNA, 38 bp, artificial sequence
  misc_feature (1)..(38): primer
gcaggctgctcatgtctguuggacgagcuucuacuggc

SEQ ID NO: 84 - DNA, 18 bp, artificial sequence
  misc_feature (1)..(18): primer
agcctcggcaacgtcagc

SEQ ID NO: 85 - DNA, 19 bp, artificial sequence
  misc_feature (1)..(19): primer
tctccttgatgggctgcag

(19) United States
(12) Patent Application Publication
(10) Pub. No.: US 2006/0035222 A1
(43) Pub. Date: Feb. 16, 2006

(54) Methods of nucleic acid amplification

(76) Inventors: Knut Rudi, Vestby (NO); Askild Holck, As (NO)

Correspondence Address: NEEDLE & ROSENBERG, P.C., SUITE 1000, 999 PEACHTREE STREET, ATLANTA, GA, 30309-3915, US

(21) Appl. No.: 10/501,632

(22) PCT Filed: Jan. 15, 2003
(86) PCT No.: PCT/GB03/00195

§ 371 Date: May 23, 2005

Related U.S. Application Data
(60) Provisional application No. 60/348,396, filed on Jan. 16, 2002.

(30) Foreign Application Priority Data
Jan. 15, 2002 (GB) 0200828.2

(51) Int. Cl. C12Q 1/68; C12P 19/34

(52) U.S. Cl. 435/6; 435/91.2

(57) ABSTRACT

The present invention provides a method of simultaneously amplifying a plurality of target sequences within sample nucleic acid which comprises: (1) contacting said sample nucleic acid with one or more primer pairs under conditions which allow hybridisation of the primers to the sample nucleic acid, each primer having a bipartite structure A-B wherein part A is specific for a particular target sequence within the sample nucleic acid and part B is a constant sequence which is common to all primers or is common amongst all forward primers with a different sequence common amongst all reverse primers; (b) performing first amplification reaction; (c) degrading the bipartite primers or separating them from the amplification products of the first amplification reaction; (d) contacting the amplification products from the first amplification reaction with primers which comprise part B of the bipartite primers or a nucleotide sequence which is substantially identical to part B, under conditions which allow hybridisation of the primers to the amplification products; and (e) performing a second amplification reaction and kits for use in such methods.

The present invention relates to methods of nucleic acid amplification, in particular to methods that employ the polymerase chain reaction (PCR).

DNA amplification techniques, and in particular the polymerase chain reaction (PCR) have become key diagnostic tools. Theoretically, a single target molecule can be detected in a background of 10¹⁰ to 10¹² non-target molecules. Recently, technology has been developed that allows nucleic acid quantification by monitoring the PCR amplification reaction in real-time (Orlando, C. P. Pinzani, and M. Pazzagli. 1998. Clin Chem Lab Med. 36(5):255-69.). There have also been efforts in the amplification of several targets simultaneously (multiplex PCR) (Elnifro, E. M., A. M. Ashshi, R. J. Cooper, and P. E. Klapper. 2000. Clin Microbiol Rev. 13(4):559-70.). This, however, is very complicated since several different primer pairs have to be optimised simultaneously.

While there is a demand in many diagnostic and other fields for multiplex PCR, the optimisation of multiplex PCR poses several problems, including poor sensitivity or specificity and/or preferential amplification of certain targets. Primers with better than average priming efficiency will produce more of their product and potentially use up the available triphosphates in the reaction mixture before amplicons relying on other less efficient primers reach detectable levels.

In addition, the presence of more than one primer pair in the multiplex PCR reaction increases the chance of obtaining spurious amplification products, primarily through the formation of primer dimers. These non-specific products may be amplified more efficiently than the desired target, consuming reaction components and producing impaired rates of annealing and extension. The optimisation of multiplex PCR should aim to minimise or reduce such non-specific interactions.

Most diagnostic assays require detection and quantification of several different targets simultaneously. The methodological limitations, are in many cases the reasons for developing simplex assays, or assays including only a few targets. This is for instance the case with the current tests for genetically modified organisms (GMOs, essentially plant material) in foods. Currently, about 50 GMO constructs are approved in for commercial use in USA. In Europe, approved GMO foods require labelling if more than 1% of any ingredient originates from a GMO. Considering the large numbers of GMOs expected in the future, multiplex quantitative measurements are required to determine whether the foods contain approved or unapproved GMO constructs, and whether the amount of GMO in the ingredients is above or below 1%.

Thus, while multiplex PCR is a very useful technique in theory, the practical problems of simultaneously performing multiple reactions are holding back its use. Recently, a technique has been proposed (Shuber, A. P., V. J. Grondin, and K. W. Klinger. 1995. Genome Res. 5(5):488-93.) which seeks to reduce the impact of the different amplification efficiencies of different primers. Such methods involve the performance of two distinct PCR reactions, with different amplification primers used in each reaction. The primers used in the first reaction are bipartite, each containing a region which is specific for a particular target sequence within the nucleic acid sample to be analysed and a universal region at the 5′ end.

Amplification cycles are performed to generate a population of amplicons from each target sequence. The region which is specific for a given target sequence hybridizes to the sample nucleic acid so that normal polymerase controlled extension can occur. The universal region does not hybridise with the original template nucleic acid but as products from earlier cycles are used as templates, this constant segment and regions complementary to it are incorporated into the amplicons. This helps to normalize the hybridisation kinetics across the different target sequences being simultaneously amplified, preventing individual target sequences being significantly over or under represented at the end of the reaction.

Then a second amplification reaction is performed using as primers oligonucleotides which comprise or consists of the universal region from the first amplification reaction. The different target regions are thus amplified using the same primers and the ratio of the number of stating molecules to end product amplicons should therefore be constant.

Such a method is described, for example in WO 99/58721 which is incorporated herein by reference. This publication particularly addresses the problems of amplifying and detecting many different target sequences in a single reaction and success is attributed to a combination of factors, including the small size of the amplification targets, optimization of amplification conditions and the presence of the constant (universal) sequence at the 5′-end of the primers.

However, in practice the methods described in WO 99/58721 and in J. Med. Genet 2000: 37 272-280, do not provide quantitative results in a multiplex PCR system. There are many scenarios where as well as testing a sample for the presence of a number of different nucleic acid sequences of interest (multiplex), it is desirable to determine the level of each sequence in a sample, i.e. to obtain quantitative results. Of particular interest is the need for food producers and food control authorities to test whether foods and food ingredients contain genetically modified plants. Already about 50 different genetically modified plants have been approved in the USA and it would clearly be very costly and time consuming to analyse a food sample for specific genetically modified plants (gmps) in a series of separate reactions. As the number of gmps increases and their use become more widespread it will be desirable to use multiplex assays to detect signature genetic elements used in gmps in a single reaction. It is also desirable to have information about whether the specific group is present in the food only in trace amounts or whether the amount is above or below a certain limit. At present there are no methods available which reliably provide this quantitative information in a multiplex environment.

A method has now been developed which addresses these problems and has been shown to provide quantitative multiplex PCR in the context of detecting gmps and which also has general applicability to assays where quantitative results of multiplex PCR are required. The method is based on the two step PCR described above but it has surprisingly been found that removal of the primers from the first amplification reaction ensures that the second amplification reaction, and thus the method as a whole, retains its quantitative character. According to this method therefore, the second amplification reaction is performed in the absence of the primers from the first amplification reaction.

Thus, according to one aspect, the present invention provides a method of simultaneously amplifying a plurality of target sequences within sample nucleic acid which comprises:
(a) contacting said sample nucleic acid with one or more primer pairs under conditions which allow hybridisation of the primers to the sample nucleic acid, each primer having a bipartite structure A-B wherein part A is specific for a particular target sequence within the sample nucleic acid and part B is a constant sequence which is common to all primers or is common amongst all forward primers with a different sequence common amongst all reverse primers;
(b) performing a first amplification reaction;
(c) degrading the bipartite primers or separating them from the amplification products of the first amplification reaction;
(d) contacting the amplification products from the first amplification reaction with primers which comprise part B of the bipartite primers or a nucleotide sequence which is substantially identical to part B, under conditions which allow hybridisation of the primers to the amplification products; and
(e) performing a second amplification reaction.

The primers used in the second amplification reaction (step (d)) will preferably be identical or substantially identical to part B of the bipartite primers used in the first amplification reaction and will typically not comprise part A or a functional part or equivalent thereof. The term ‘substantially identical’ will be understood with functional considerations in mind, i.e. the ability to hybridise efficiently to the amplification products of the first amplification reaction. Typically this will mean no more than 5 nucleotide additions, deletions or substitutions, preferably no more than 3. These primers ‘comprise part B of the bipartite primers (or a nucleotide sequence which is substantially identical to part B)’, i.e. the nucleotide sequence of these primers comprises the same sequence as part B of the bipartite primers used in the first amplification reaction (or a nucleotide sequence which is substantially identical to part B).

In a preferred embodiment, the constant region B of the bipartite primers is common between both forward and reverse primers and thus only a single primer species is required in the second amplification reaction. In an alternative embodiment, it may be desirable to have different forward and reverse primers, with one of the primer species labelled for subsequent detection. Whether the constant region (B) is common to all primers or only amongst the forward or reverse primers, it is found at the 5′end of both the forward and reverse primers; the variable section (A) which is designed to hybridise to a sequence in the sample nucleic acid is found at the 3′end of the bipartite primers. Thus the abbreviation ‘A-B’ does not imply a relative position within the molecule for the two regions in terms of the 3′ and 5′ ends. The constant region (B) is typically 10-40 nucleotides in length, preferably 12-25 nucleotides in length.

The region B will either be substantially the same in all bipartite primers or substantially the same amongst the forward primers with a second region B′ which is different to B but is substantially the same amongst all the reverse primers. Preferably B (or B′) will be exactly the same in all bipartite primers or at least in all forward or all reverse primers but it will be understood that a small number of nucleotide variations between sequences will not significantly affect the method. The term ‘common’ should be interpreted with this in mind. The purpose of these constant regions is to even out differences in priming efficiency and to provide highly efficient hybridisation and priming with the primers used in the second amplification reaction. Therefore between B sequences which are substantially the same there will preferably be variation at no more than 3 nucleotide positions.

Preferably the constant region(s) B is chosen so that it does not hybridise with the sample nucleic acid, or at least does not hybridise efficiently therewith. Thus a randomly chosen sequence may be constructed according to the well known rules for primer design.

Part A of the bipartite primers is specific for particular target sequences in that they are designed to hybridise to a region of nucleic acid which flanks the target sequence which it is desired to amplify. According to the normal conventions of the PCR, the A sequences will be in pairs, each pair consisting of a forward primer and a reverse primer which hybridise to regions upstream and downstream of a nucleotide sequence of interest. The bipartite primers will therefore be formed into pairs of forward and reverse primers by the nature of their A sequence. In certain cases, part A may be substantilly identical in the forward and the reverse primer, for example when the desired target sequence is flanked by inverted repeats as is often the case with mobile elements such as transposons.

The primers may have the form A^{F1}-B, A^{R1}-B, A_{R1}-B, A^{R2}-B etc. where ‘A^{F1}’ indicates a forward primer sequence which hybridises to a flanking region of a first target sequence and ‘A^{R1}’ a reverse primer sequence which hybridises to the other flanking region of the first target sequence. As mentioned above, the common regions B may be different in forward and reverse primers, thus having the form A^{F1}-B, A^{R1}-B′, A^{F2}-B, A^{R2}-B and so on.

The part A regions which hybridise to specific-regions in the sample nucleic acid amplification are selected by methods well known in the field of nucleic acid amplification. In order to select a pair of A sequences for amplifying a target region, the sequence of and adjacent to the target sequence must be known (or at least approximately known). Short stretch sequences at either end of the target sequence are then selected and the primers designed for hybridisation to these regions.

Typically only a few cycles will be performed in the first amplification reaction, e.g. less than 25, preferably less than 15, more preferably less than 10, to avoid potential artefacts in the multiplex amplification and to ensure that none of the targets reach saturation levels. Preferably this first amplification reaction is carried out using standard PCR reagents and conditions and suitable parameters for the cycles are described in the examples and are generally well known in the art. The ‘first’ and ‘second’ amplification reactions therefore refer to two sets of amplification cycles, each defined by the primers involved.

To increase amplification efficiency for a given target sequence, the primer concentrations for that target may be increased for the first amplification reaction.

The bipartite primers are then separated from the amplification products of the first amplification reaction before the second amplification reaction takes place. By ‘separation’ is meant the separation of the bipartite primers and the amplification products into two distinct pools, not the dissociation of primer and template which occurs as an integral part of all standard PCR reactions. This may be achieved by removing the bipartite primers, conveniently this is done by breaking down the bipartite primers e.g. by exonuclease degradation. Alternatively, the bipartite primers could possess a non-standard modification, e.g. contain uracil instead of thymine, and could therefore be degraded by a DNA-modifying enzyme such as uracil-DNA glycosylase. This enzyme removes uracil from the sugar backbone which leads, on heat treatment, to a strand break. This enzyme removes uracil from the sugar backbone which leads, on heat treatment, to a strand break. The use of bipartite primers which contain uracil only in the A part would allow the selective degradation of only this part, leaving part B intact, which could then participate in the second amplification reaction.

Thus, reference above to ‘degradation’ of the bipartite primers includes both full or partial degration and so a molecule which has been partially broken down can be considered to be degraded. It is important that the A parts of the bipartite primers are no longer available to take part in hybridisation reactions during the second amplification reaction. A ‘DNA-modifying enzyme’ is therefore able to inactivate the bipartite primers or at least part A thereof.

Alternatively the amplification products may be isolated from the rest of the initial reaction mixture which contains the bipartite primers. The products of the first amplification reaction are thus purified before being used as templates for the second amplification reaction. Purification is conveniently achieved by capturing the amplification products on a solid support e.g. by using a standard PCR product purification kit or through attaching a binding moiety to the amplification products and providing a binding partner for said binding moiety on the solid support. The binding moiety may be attached to a probe which in turn hybridises to the amplification product. Suitable binding moieties are well known in the art and include, streptavidin/biotin, antigen/antibody interactions, lectin binding systems or probes covalently bound to a solid support etc. Suitable solid supports are also well known and widely available, preferably the support is magnetic and particulate for ease of manipulation.

Key to step (c) is the fact that all or most, i.e. at least 70%, preferably at least 80%, more preferably at least 90% of the bipartite primers are degraded or separated from the amplification products before the second amplification reaction takes place.

The second amplification reaction uses either a single primer species or a single forward primer species and a single reverse primer species. If appropriate, these may be present in the reaction mix from the start, i.e. during the first amplification reaction, or be generated through the modification or partial degradation of the bipartite primers, for example as described above where uracil replaces thymine in part or all of the bipartite primers. In other embodiments of the invention, e.g. where an exonuclease is used to degrade the primers or the amplification products are separated from the bipartite primers through the use of a labelled probe, the primers used in the second amplification reaction will not be present in the initial reaction mix. Step (d) of the method defined above therefore encompasses all of these variants.

It may be desirable to perform all the method steps (i.e. both the first and second amplification reactions) in one reaction vessel and degradation of the bipartite primers may conveniently allow this.

The advantages of the methods of the invention are twofold. One limitation of multiplex PCR is the different amplification efficiencies of the different amplicons when specific primer sets are used. This will lead to a situation where some of the amplicons present are amplified whereas others are not. In addition, using many different primer pairs in one reaction inevitably leads to a large number of side reactions due to primers interacting with each other. These side reactions perturb the PCR. The use of a constant part B in the first step primers in combination with the removal of those primers eliminates these problems. Secondly, the amplification of all targets with the same primer or primer pair leads to a constant ratio of the different targets in the multiplex PCR before and after amplification, in the same way as in competitive PCR. By effectively removing the bipartite primers after the first PCR step, these do not interfere with the ratios of the different amplicons during the second PCR step. This removal is what makes the system maintain its quantitative nature.

‘Amplification’ refers to a process for using polymerase and a pair of primers for increasing the amount of a particular nucleic acid sequence, a target sequence, relative to the amount of that sequence initially present in the sample nucleic acid. Amplification may conveniently be achieved by the in vitro methods of PCR (including reverse transcriptase PCR (RT-PCR)) or ligase chain reaction or others as well as NASBA (nucleic acid sequence based amplifications) approaches.

A ‘target sequence’ is a sequence that lies between the hybridisation regions of a pair of primers (and may in addition include the primer sequences themselves) and can be amplified by them. The number of different target sequences within the sample which may be amplified will depend on the nature and requirements of the assay. Typically there will be more than 4, e.g. 8 or more even 12 or 20 or more different target sequences amplified in one multiplex reaction.

In the context of assaying for the presence of GMOs, the target sequences may fall into one of a number of categories. The target sequence may fall entirely within a gene of interest and the ampicillin PCR in the multiplex system described in the present Examples is an example of this. The ampicillin resistance gene is included in pUC18 which is used in the generation of Bt176 corn (Maximizer Corn). A positive PCR result shows the presence of the gene but does not determine the origin of the DNA and therefore the amp signal could originate from Bt176 DNA but could also originate from a bacterial contamination of the plant.

A gene of interest is typically part of a construct of interest and a promoter often used in such constructs is the 35S promoter from the Cauliflower mosaic virus (CaMV). One of the PCRs in the multiplex PCR described in the present examples detects this promoter and thus target sequences may be in regulatory regions. Although again, a positive result may indicate that the plant has been infected with Cauliflower mosaic virus. The nos reaction of the present examples detects a different regulatory region used in these constructs, the NOS terminator.

A more specific approach is to design a primer pair overlapping a junction region between a promoter or terminator (a regulatory region) and a gene of interest. These DNAs do not occur naturally in nature and thus a PCR signal would be a very strong indication of the presence of GMOs. In the present examples such an overlap is detected in the multiplex PCR system for Bt176 and Bt11 (Methods for the specific detection of Bt176 corn and Bt11 corn are described in Hurst, C. D. et al. (1999) European Food Research and Technology Vol. 5, 579-586 and Zimmermann, A. et al. (2000) Lebensmittel-Wissenschaft & Technologie, 33, 210-216 respectively). In the Bt176 PCR a fragment overlapping the junction between the pepC promoter (phosphoenol pyruvate carboxylase promoter from maize) and the cry gene (a synthetic gene from Bacillus thuringiensis which confers insect resistance) is targetted. In Bt11 the PCR overlaps the junction between the 35S promoter and an enhancer DNA fragment from the alcohol dehydrogenase gene from maize. In a preferred embodiment of the present invention, one or more of the target sequences spans a non-naturally occurring nucleic acid sequence, e.g. a sequence comprising regions which are not naturally found in juxtaposition.

However, even this approach could conceivably cause problems if a company used the same construct, e.g. a specific promoter-enhancer-gene-terminator in several different plants (be it the same species or not, but different transformation events). One of these transformations (GMOs) may be approved by the relevant regulatory body while others are not but the PCR would not be able to discriminate between the approved GMO and the non-approved GMO(s). When a plant is transformed, DNA integrates randomly at different sites for each transformation event. Thus a way of overcoming the problems discussed above would be to determine the plant DNA sequence which flanks the inserted DNA, and then construct a primer pair which overlaps this junction (which can be called an ‘event specific region’).

Thus in a preferred embodiment of the methods of the present invention one or more of the target sequences is for an event specific region, i.e. spans a region which comprises both host plant species DNA and inserted DNA from the genetically engineered construct.

The Mon810 PCR of the present examples is an example of such an event specific region (Zimmermann et al. (1998)) Food Science and Tech. 31, 664-667 have designed a nested PCR system for the detection of Maisgard corn (Mon810 corn) as the amplified sequence lies in the overlap between integrated DNA and the plant's endogenous DNA.

The sample nucleic acid may be isolated or may exist as part of a mixed sample which includes other cellular components from the biological source from which it was obtained. Methods of isolating nucleic acid from a biological sample are well known in the art. Any biological sample containing nucleic acid is a suitable source of nucleic acid and thus the sample may be derived from animals, plants, insects, bacteria, yeast, viruses or other organisms. Particularly preferred sources of sample nucleic acid for amplification according to the present invention are plants or food products which contain or are suspected of containing genetically modified material. The ‘sample nucleic acid’ may be derived from one or more biological samples. In the context of plants and foodstuffs for example, a single plant may provide the sample nucleic acid or it may be derived from a number of plants of the same or even different species.

By ‘nucleic acid’ is meant DNA (including cDNA) or RNA. The nucleic acid may be naturally occuring or synthesised by chemical or recombinant techniques.

The above amplification method is then generally followed by a detection step and suitable detection methods for multiplex PCR are known in the art and discussed, for example, in WO 99/58721. When performing a multiplex reaction it is necessary to differentiate between the amplification products from different loci. This could be done on the basis of size discrimination, e.g. on gels but requires the amplification products to be of different sizes, e.g. 100 bp, 200 bp etc. The reaction products could be differentially labelled, i.e. different tags are attached to primers for different loci, however such a technique is limited by the number of different commercially available tags (e.g. fluorescent molecules).

Thus in a preferred embodiment probes specific to the different nucleotide sequences of interest which have been amplified are enzymatically labelled at their 3′end and then the labelled probes are captured by hybridisation to complementary DNA on a solid support e.g. nylon filters, glass slides, chips etc. Such methods are described in the Examples and in WO 99/50448.

These probes to the different target regions may be labelled at the 5′-end with a fluorescent group other than the one used in the 3′-end labelling reaction. During fluorescent scanning it would then be possible to calculate immediately the percentage of molecules labelled during the labelling reaction.

As discussed above, the methods claimed herein are quantitative in nature. The signal strengths for identified target sequences can be compared to known standards to calculate the concentration (e.g. copy number) of that target sequence in the sample. As described in the Examples, a known concentration of a control sequence (IPC) may conveniently be added to the sample to adjust for fluctuations in amplification efficiency from one sample reaction mix to another; the use of such an internal control determines the absolute amount of nucleic acid in the sample and is a preferred embodiment of the present invention. In a further preferred embodiment, also described in the Examples, a species specific target sequence is amplified and this reference gene enables the relative amounts of nucleic acid constructs/sequences of interest (e.g. a target GM construct) as compared to the material from said species to be determined.

Thus, the invention provides data for a given target sequence which can be quantified against a known reference for that target sequence. Target sequences can be detected qualitatively and quantitatively according to the methods of the invention and the results from different experiments compared because quantifiable information is obtained.

In a further aspect, the present invention provides a kit for use in a method of nucleic acid amplification, typically any method as described above, which comprises:
(a) a plurality of bipartite primer pairs of form A-B as defined above;
(b) means for degrading the bipartite primers or for separating them from the amplification products of a first amplification reaction; and optionally
(c) primers which comprise part B of the bipartite primers of component (a) or a nucleotide sequence which is substantially identical to part B of said primers.

Means (b) may conveniently include exonucleases which degrade the primers, standard PCR-product purification kits or probes that capture the amplification products on a solid support. Where part A but not part B of the bipartite primers contains thymine, means (b) may conveniently comprise an enzyme such as uracil DNA glycosylase which selectively degrades part A of the bipartite primers, generating primers for use in a further amplification reaction. In which case, a separate component (c) may not be required.

The invention will be further described in the following non-limiting examples and with reference to the Figures in which:

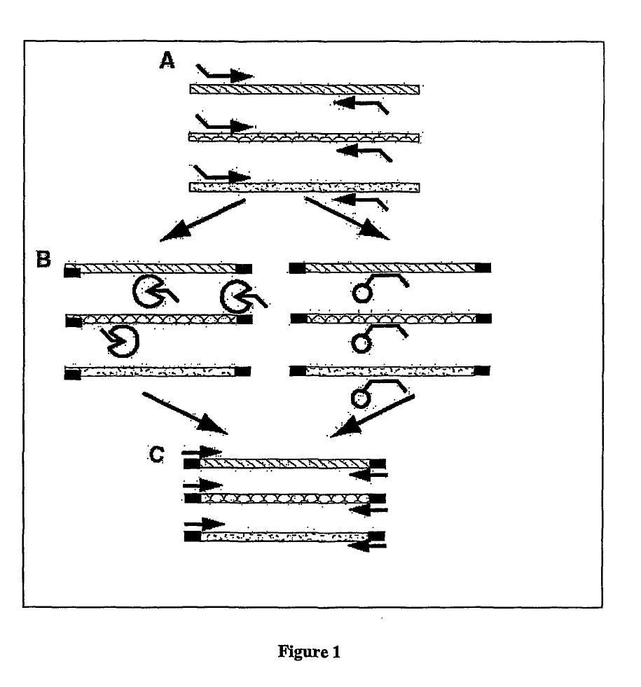

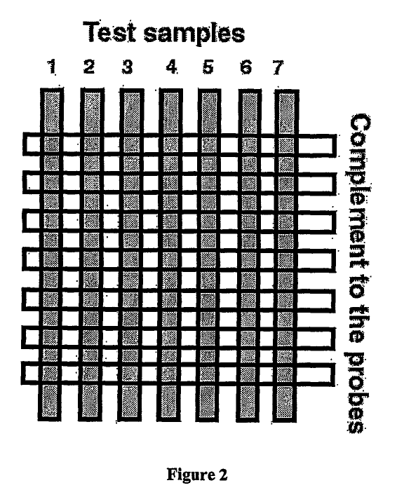

FIG. 1 provides a schematic representation of the quantitative multiplex amplification method. (A): In the first PCR step, the targets are amplified with primers containing “heads” that are equal for all the targets. (B): The “head”-containing primers are then removed by enzymatic digestion (left) or the amplified products are hybridized to an internal biotinylated capture probe and the complex is then purified through binding to biotinylated paramagnetic beads (right). These are two independent alternative purification strategies. (C): In the second PCR step, a primer identical to the “head” sequence is used.

FIG. 2 provides a schematic illustration of the test format. The probes complementary to the labelled test probes used in the enzymatic labelling are spotted horizontally using a grid. During hybridisation the grid is turned 90 degrees before application of hybridisation solutions and labelled probes.

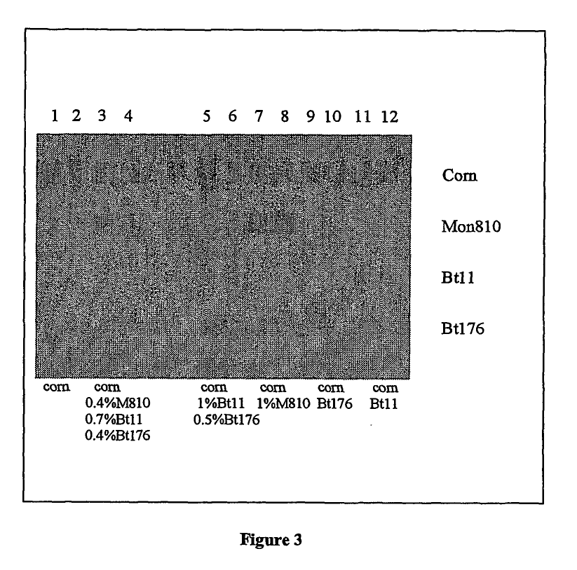

FIG. 3 shows multiplex detection of GMO corn samples. GM corn DNA was analysed either alone or in combinations. Line 1: detection of the corn reference DNA, line 2: Mon810 signals, Line 3: Bt11 signals, Line 4: Bt176 signals. The samples analysed are indicated under the corresponding lanes (all analyses in duplicate), Lane 1,2: non GMO maize, lane 3, 4: 0.4% Bt176, 0.7% Bt11 and 0.4% Mon810 DNA, lane 5, 6: 1% Bt11 and 0.5% Bt176, lane 7, 8: 1% Mon810, lane 9,10: 1% Bt176, lane 11, 12: 2% Bt11.

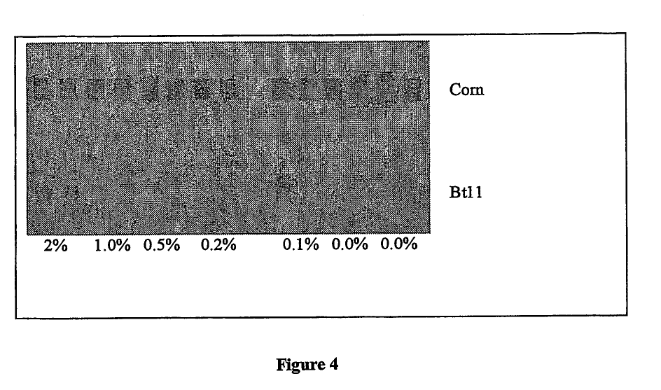

FIG. 4 shows quantitative chromogenic detection of Bt11 corn DNA using the multiplex assay. 2% Bt11 corn DNA was diluted in non GM corn DNA to give different concentratons of GM corn. The results show the quantitative response of the assay as the concentration of GM corn is lowered. The first line shows the corn DNA reference signals, the second row shows the Bt11 signals. The signals were recorded on a Typhoon scanner, PE systems.

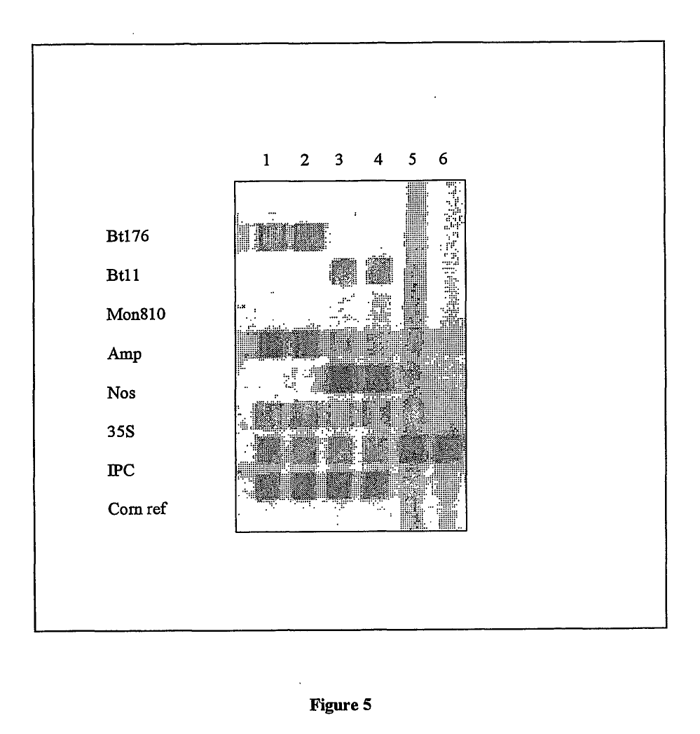

FIG. 5 shows eight-plex detection of GM maize. Eight specific primer pairs with “heads” were used in the first PCR step. The lines represent (from above): Bt 176, Bt11, Mon810, amp, Nos terminator, 35S promoter, Internal PCR control (IPC) and maize reference gene. Lanes 1 and 2: 2% Bt 176 maize DNA, lanes 3 and 4: 1% Bt11 maize DNA, lanes 5 and 6: 1 PC.

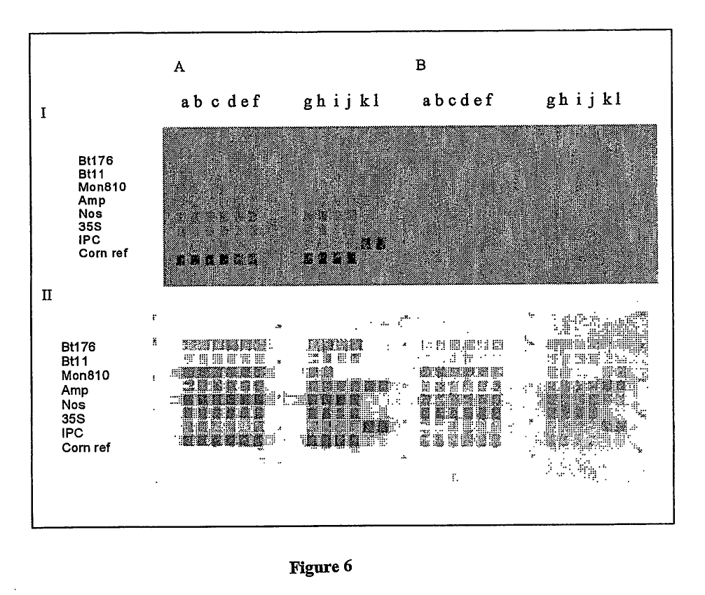

FIG. 6 shows quantitative multiplex PCR for detection of GM corn and the necessity of removing primers after the 1. PCR step. Each line shows the detection of a specific PCR product as indicated to the left. Each lane (a through 1) represent different samples. All samples (a-j) contained a mixture of 0.7% Bt176 and 0.7% Bt11. In addition Mon 810 corn DNA was added to 2.0% (lanes a, b), 1.0% (lanes c, d), 0.5% (lanes e, f), 0.2% (lanes g, h) and 0.0% (lanes i, j). In addition all lanes (a-1) contained approx. 100 copies of an internal positive control (IPC) DNA. Amp: ampicillin resistance gene from the pUC18. Nos: Nos terminator, 35S: Cauliflower mosaic virus promoter.

(A): PCR carried out in two steps: 1. PCR (10 cycles) using specific primers with a common “head” sequence. Primers are then digested and the 2. PCR (30 cycles) is carried out using the common head primer. (B) Same as A, but the specific primers were not degraded before the 2.PCR step. Panel I: shows the fluorescence signals after hybridisation and scanning, panel II: shows the blot after binding of antibodies and enzymatic HRP colour reaction.

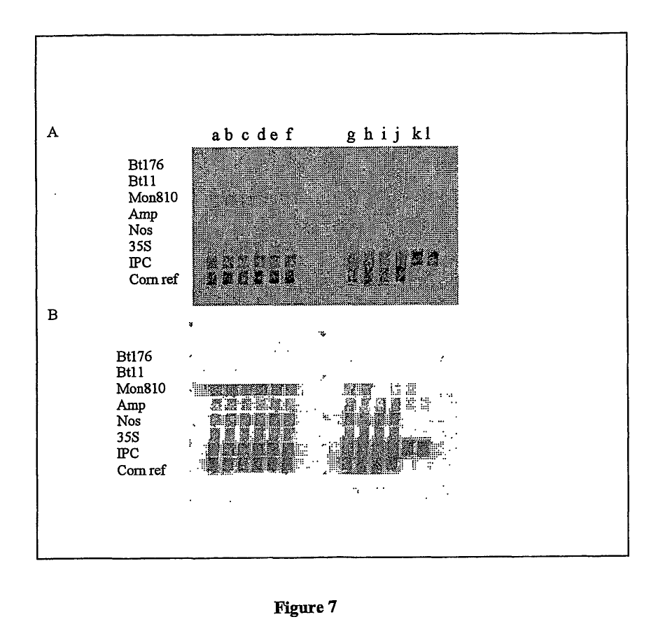

FIG. 7 illustrates the effect of omitting the 2. PCR step. Same as in FIG. 6A, except that the 1.PCR step using the specific primers with head sequence was extended to 40 cycles and the 2. PCR step was omitted. Panel A shows the fluorescence signals after hybridisation and scanning, panel B shows the blot after binding of antibodies and enzymatic HRP colour reaction.

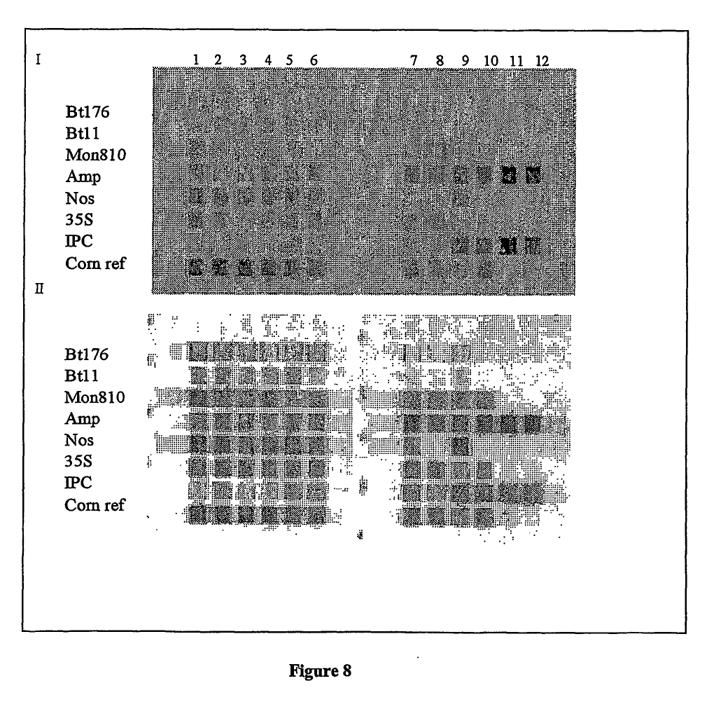

FIG. 8 illustrates the effect of diluting the template DNA. A reference mixture of 0.7% Bt176, 0.7% Bt11 and 0.7% Mon810 at different dilutions was used as templates in the PCR. Panel I shows the fluorescence signals after hybridisation and scanning, panel II shows the blot after binding of antibodies and enzymatic HRP colour reaction. Lanes 1, 2: undiluted DNA template, lanes 3, 4: 1/4 dilution, lanes 5, 6: 1/16 dilution, lane 7, 8: 1/64 dilution, lanes 9, 10: 1/256 dilution, lanes 11, 12: no template added.

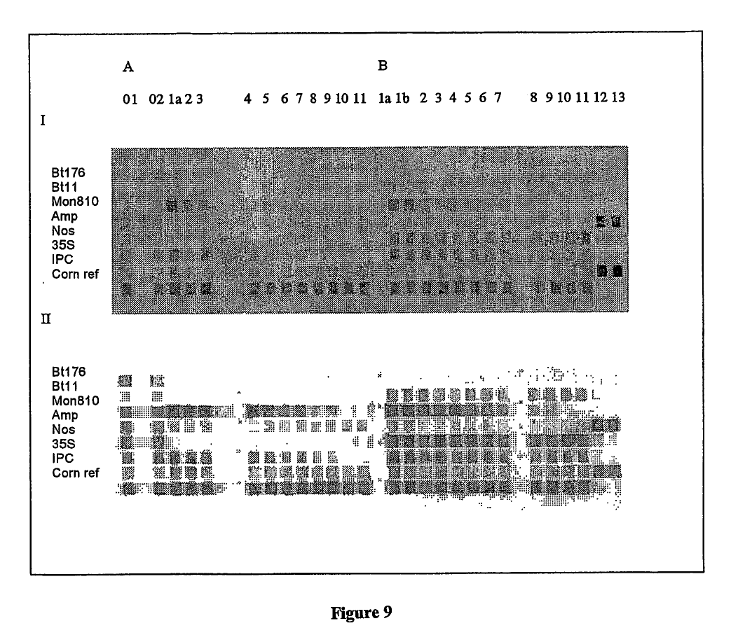

FIG. 9 shows quantitative 8-plex detection of Mon810 DNA alone (A) or together with 2% Bt11 DNA (B). Panel I shows the fluorescence signals after hybridisation and scanning, panel II shows the blot after binding of antibodies and enzymatic HRP colour reaction. Lanes 01, 02: A reference mixture of 0.7% Bt176, 0.7% Bt11 and 0.7% Mon810. Lane 1a, 1b: 5% Mon810, lanes: 2, 3: 2% Mon810, lanes 4, 5: 1.0% Mon810, lanes 6, 7: 0.5% Mon810, lanes 8, 9: 0.1% Mon810, lanes 10, 11: 0% Mon810, lanes 12, 13: IPC (date 020901).

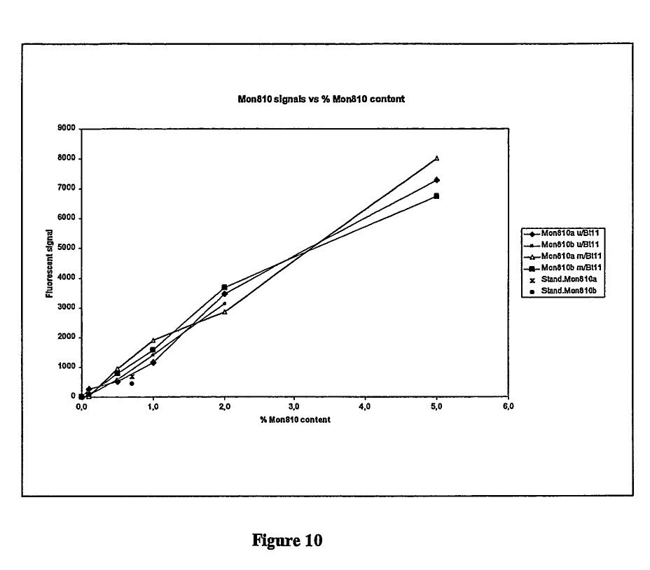

FIG. 10 shows the relationship between amount of Mon810 maize in a sample and the signal strength. The Mon810 fluorescence signals in FIG. 9 panel I, were quantified using Imagemaker program and plotted against the given concentration of the samples.

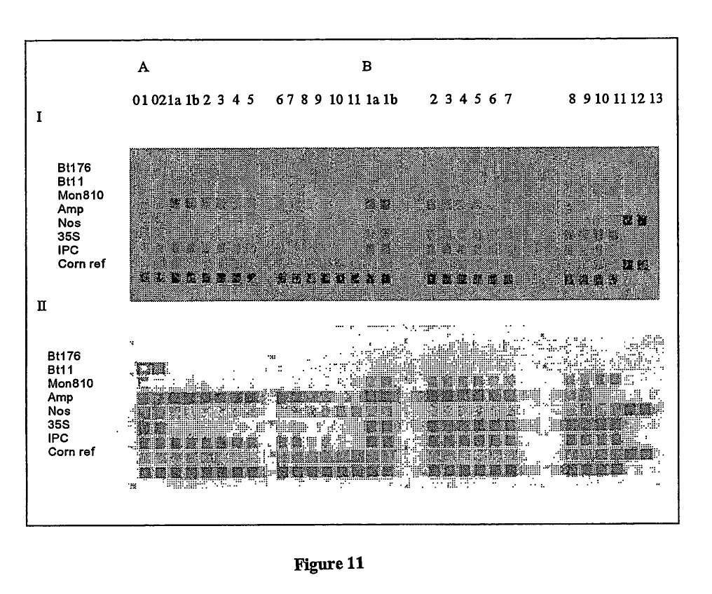

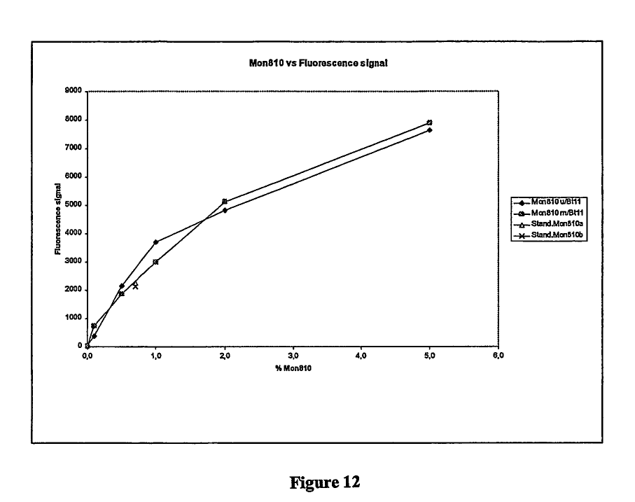

FIG. 11 illustrates quantitative 8-plex detection of Mon810 DNA alone (A) or together with 2% Bt11 DNA (B). Repetition of example 6 (FIG. 9). Panel I shows the fluorescence signals after hybridisation and scanning, panel II shows the blot after binding of antibodies and enzymatic HRP colour reaction. Lanes 01, 02: A reference mixture of 0.7% Bt176, 0.7% Bt11 and 0.7% Mon810. Lane 1a, 1b: 5% Mon810, lanes: 2, 3: 2% Mon810, lanes 4, 5: 1.0% Mon810, lanes 6, 7: 0.5% Mon810, lanes 8, 9: 0.1% Mon810, lanes 10, 11: 0% Mon810, lanes 12, 13: IPC (date 130901).

FIG. 12 shows the relationship between amount of Mon810 maize in a sample and the signal strength. The Mon810 fluorescence signals from the experiment in FIG. 11 were quantified using Imagemaker program and plotted against the given concentration of the samples. The average of 2 parallels are shown.

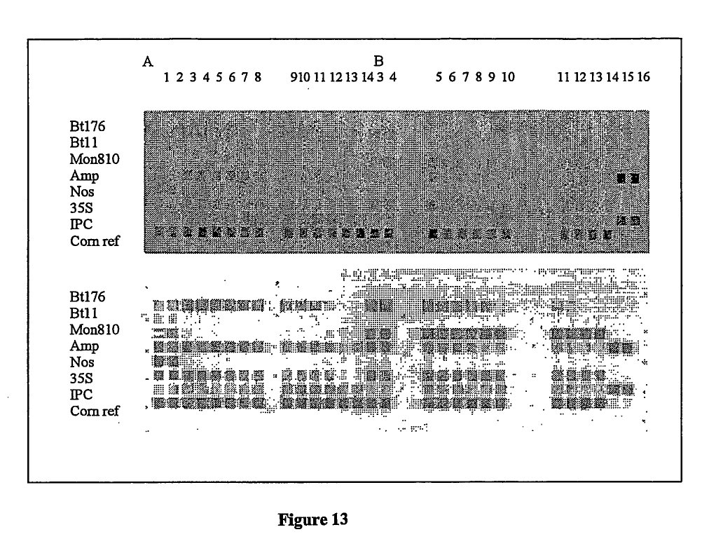

FIG. 13 illustrates quantitative 8-plex detection of Bt176 DNA alone (A) or together with 1% Mon810 DNA (B). Panel I shows the fluorescence signals after hybridisation and scanning, panel II shows the blot after binding of antibodies and enzymatic HRP colour reaction. Lanes 1, 2: A reference mixture of 0.7% Bt176, 0.7% Bt11 and 0.7% Mon810. Lane 3, 4: 2% Bt176, lanes: 5, 6: 1% Bt176, lanes 7, 8: 0.5% Bt176, lanes 9, 10: 0.2% Bt176, lanes 11, 12: 0.1% Bt176, lanes 13, 14: 0% Bt176, lanes 15, 16: IPC (date 060901).

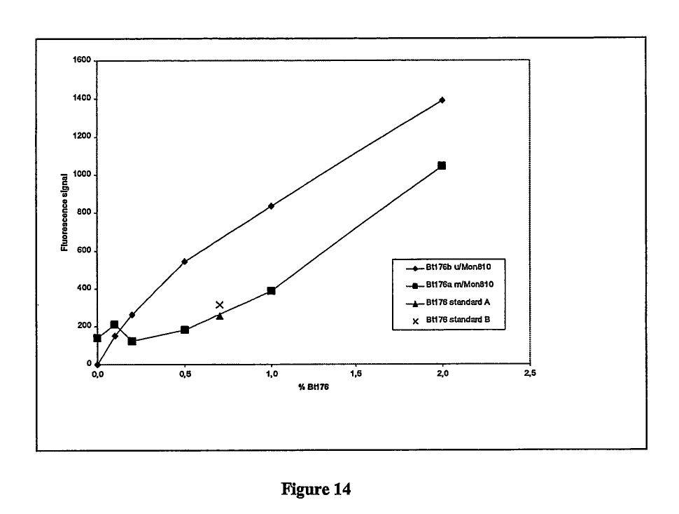

FIG. 14 shows the relationship between amount of Bt176 maize in a sample and the fluorescence signal strength. The Bt176 fluorescence signals from the experiment in FIG. 13 were quantified using Imagemaker program and plotted against the given concentration of the samples. The average of 2 parallels are shown.

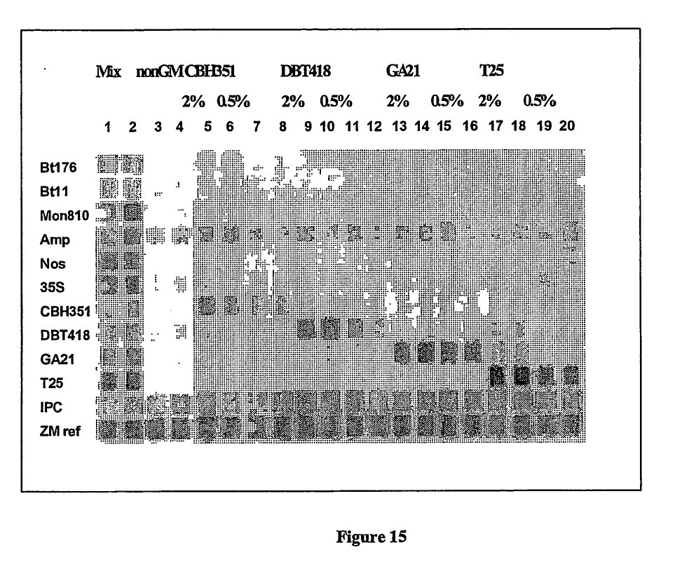

FIG. 15 Twelve-plex system for detection of seven different GM maize events. HRP enhanced chromogenic signals are shown. Samples 1, 2: a mixture of 0.7% of each of Mon810, Bt11 and Bt176 and 1% of each of T25, GA21, CBH351 and DBT418, 3, 4: non-GM maize, 5, 6: 2.0% CBH351, 7, 8: 0.5% CBH351, 9, 10: 2% DBT418, 11, 12: 0.5% DBT418, 13, 14: 2% GA21, 15, 16: 0.5% GA21, 17, 18: 2% T25, 19, 20 0.5% T25. Amplicons are as described for the eight plex PCR in FIG. 9. with additions of amplicons for CBH351, DBT418, GA21 and T25 given in Table 1.

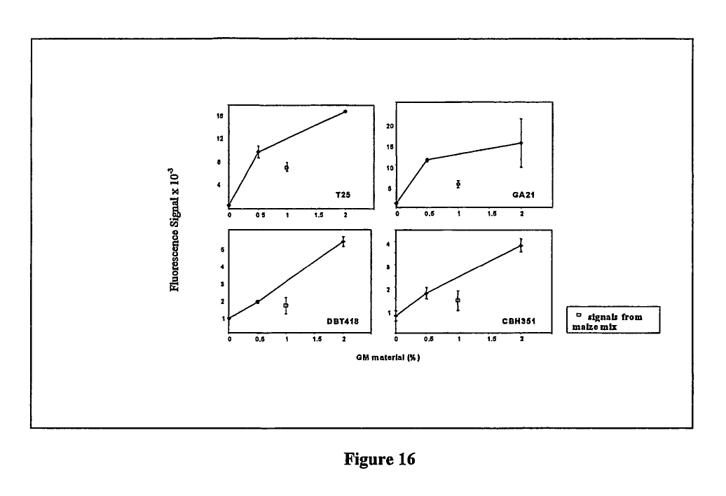

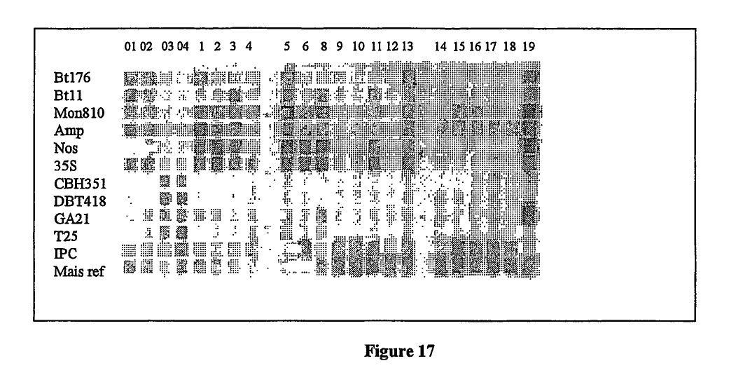

FIG. 16 Twelve-plex system for detection of seven different GM maize events. Quantifications of the fluorogenic signals for CBH351, DBT418, GA21 and T25 from the experiment shown in FIG. 15. (□) Signals obtained from samples 1 and 2 of FIG. 15.

Example 17 Screening of commercial samples using the 12-plex PCR system. The results after chromogenic enhancement is shown. Samples 01, 02: Reference mix containing 0.7% of each of Bt176, Bt11 and Mon810, 03, 04: Reference mix containing 2% of each of CBH351, DBT418, GA21 and T25. 1-19: Food and feed samples. All samples contained approx. 100 copies of IPC (internal positive control).

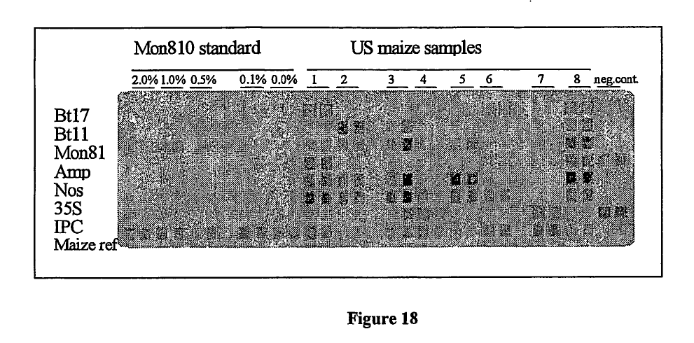

FIG. 18 Quantification of food and feed samples from USA with regards to Mon810 using the eight-plex PCR system. Samples are as indicated in the Figure. US maize sample no. 4 is an additional maize meal reference containing 1% Mon810. All samples were analysed in duplicate.

Example 19 Quantification of food and feed samples from USA with regards to Bt11 and Bt 176 using the eight-plex PCR system. Samples are as indicated in the Figure. All samples were analysed in duplicate.

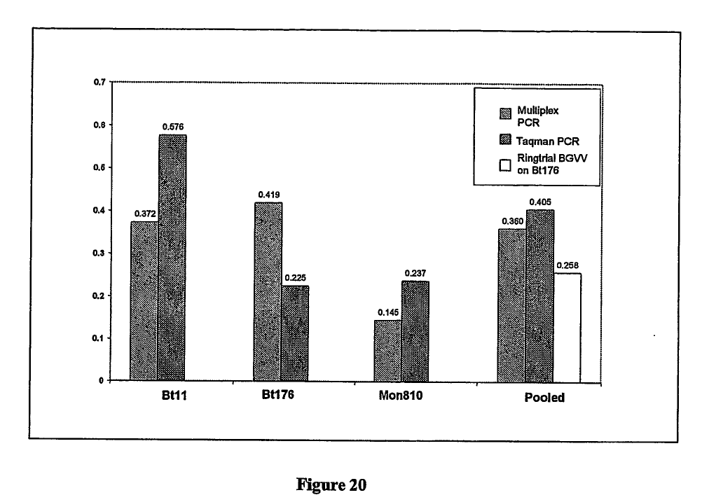

FIG. 20 Comparisons of standard deviations for multiplex PCR and 5′nuclease PCR on food and feed samples from USA per. Yellow column: averaged standard deviation for 5′ nuclease PCR in a European ringtrial for determination of Bt176. The ringrial included six maize meals analysed by nine different laboratories.

### EXAMPLES

Materials and Methods

Template and DNA Purification.

The method chosen exploits the use of DNA adsorption columns provided by Qiagen in the DNeasy plant mini kit. Samples were homogenised when necessary and purified as described by the manufacturer with the following modifications.

The initial buffer volume was doubled and lysis was carried out for 30 min at 65° C. using a shaking incubator. When eluting DNA bound to the column, 50 μl of preheated buffer was used. In the repeated elution step another 50 μl buffer was added and the columns were spun at 13000 rpm for 2 min.

The criteria used for assessing the quality of the DNA preparation was that no inhibition should be detected when samples were analysed with different BioInside kits. This is easily seen on the internal PCR Control (IPC) provided by BioInside. Also the quality of DNA was analysed carrying out PCR on dilutions of a sample and calculating the amplification efficiency and quantifiable range of the PCR by plotting the Ct values against the log DNA concentration and performing linear regression analysis. A large number of different food samples (>100) have been analysed giving good results with this DNA purification method.

The maize reference gene used herein is the maize zein gene.

PCR amplification. Purified DNA was used in the amplification reactions. We used a two step PCR amplification approach (see FIG. 1 for a schematic representation). In the first step we used primers with both a 5′-universal “head” and a gene specific region (see Table 1 below which shows the specific regions of the bipartite probes, the biotin labelled isolation probe (as this may be used in place of degradation to separate primers from amplification products) and the GM specific probes which are labelled and then take part in DNA array hybridisation).

Primers with a “head” were then removed by enzymatic degradation or by transfer of the PCR products to new tubes by capturing DNA onto paramagnetic beads labelled with specific capture probes. In the second PCR step a primer identical to the universal “head” region was used.

In the first PCR step we used 10 pmol of each of the primers, 1× Dynazyme DNA polymerase reaction buffer, 10 mM dNTP, and 2 μl DynaZyme DNA polymerase (2U μl) in a final volume of 50 μl. In some cases (for Bt11 detection) the concentration of primers was increased. The amplification protocol used was as follows (1.PCR step); 4 cycles using the parameters 95° C. for 30 s, 55° C. for 30 s, and 72° C. for 30 s, and then 6 cycles using the parameters 95° C. for 30 s and 72° C. for 30 s. Twenty μl of the amplification product from PCR step 1 were treated with 2 μl Exonuclease I to degrade the residual single stranded primers, and 3 μl shrimp alkaline phosphatase to inactivate the nucleotides. The reaction was incubated at 37° C. for 30 min, and then at 95° C. for 10 min to inactivate the added enzymes.

Five μl of the exonuclease treated products were then used for the second PCR amplification step. 50 pmol of a universal primer identical to the universal region (“head”) of the primers used in the first PCR reaction were added. The other components were the same as in the first amplification. The amplification conditions used were: 95° C. for 15 s and 70° C. for 45 s for 40 cycles. During the course of the work some changes and modifications in the PCR conditions were adopted. In the introductory experiments the 2. PCR step was carried out under the following conditions: 40 cycles of 95° C. for 15 s, 65° C. for 15 s and 72° C. for 30 s. Later (pertaining to FIGS. 3, 4 and 5): the conditions were changed to 95° C. for 15 s and 70° C. for 45 s for 40 cycles. In later experiments the same conditions were used but the number of cycles were reduced to 30. In the final experiments the number of cycles in the 1.PCR was reduced to 4, and the number of cycles in the 2. PCR step again increased to 40.

Sequence Specific Labelling.

After amplification with the “head” primer the amplification products were treated with 2 μl exonuclase 1 and 2 μl shrimp alkaline phosphatase at 37° C. for 30 min, and then 95° C. for 10 min to inactivate the enzymes. The cyclic labelling conditions were as follows; 1× Thermosequenase reaction buffer, 10 pmol of each GM specific probe, 100 pmol ddNTP (except ddCTP), 100 pmol Fluorescein-12-ddCTP, 16 U Thermosequenase DNA polymerase, and 24 μl phosphatase and exonuclease treated PCR product. The labelling was done using the following parameters; 95° C. for 15 s, 60° C. for 1 min for 15 cycles, 95° C. for 15 s, 55° C. for 1 min for 15 cycles, and finally 95° C. for 15 s, 50° C. for 1 min for 15 cycles.

DNA Array Hybridisation.

The format of the assay is shown in FIG. 2. 400 pmol/500 μl probes complementary to those used in the labelling reaction were spotted on Gene screen Plus nylon membranes (NEN), and crosslinked for 15 min with a UV transilluminator (Model TL33, UVP Inc., San Gabriel, Calif.). The membranes were prehybridized in 0.5 M Na₂HPO₄ pH 7.2 and 1% SDS for 2 hours. The labelled probes were added to 300 μl of 1×SSC and 6% PEG 1500 heated to 80° C. for 5 min. The hybridisation was done over-night at room temperature with agitation in a Cross Blot Dot Blot hybridisation chamber (Sebia, Moulinaux, France). The membrane was subsequently rinsed in 1×SSC, 1% SDS for 5 min, then 5 min in 0.1×SSC, 0.1% SDS, and finally 5 min in 0.1 M Tris-HCl pH 7.5 and 0.15 M NaCl (antibody buffer). At this point the fluorescence was detected directly using a Typhoon scanner (Amersham-Pharmacia). The membranes were then blocked in for 1 hour in blocking buffer: antibody buffer containing 1% skimmed milk (Difco, Detroit, Mich.). Blocking buffer containing 1/500 antifluorescein HRP-conjugate was then added, and the hybridisation continued at room temperature for 1 hour. Finally, the membranes were rinsed for 30 min in antibody buffer, and the signals detected with 4 CN Plus chromogenic substrate according to the manufacturers recommendations (NEN).

Quantification of scanned signals was carried out using the Imagemaster™ Array software version 2.0 program and calculations were done with Microsoft Excel 97 SR-2.

TABLE 1
Primers and probes used in the PCR reactions

| | |
|---|---|
| Primer/ | |
| probe name | Sequence (5′-3′) |

| | |
|---|---|
| Head- | 5′- TGC TAT GCG CGA GCT GCG - 3′ |
| sequence | |

| | | | |
|---|---|---|---|
| Mon 810 | | | |
| Head | Forward | Mon810F1101MH | AAT AAA GTG ACA GAT AGC TGG GCA |
| primer | | | |
| | Reverse | Mon810R1101MH | CCT TCA TAA CCT TCG CCC G |
| Biotin | | Mon810 HH | |
| labelled | | | TTT TTA CGA AGG ACT CTA ACG TTT AAC ATC |
| probe | | | CTT TGC CAT TTT T |
| Mud F | | Mon810 Mud1101 | ACG AAG GAC TCT AAC GTT TAA CAT CCT TTG C |
| Mud R | | Mon810 MudCap | GCA AAG GAT GTT AAA CGT TAG AGT CCT TCG T |
| Bt11 | | | |
| Head | Forward | AHJ-2MH | CGC ACA ATC CCA CTA TCC TT |
| primer | | | |
| | Reverse | Bt11 RDMH | GCC TCC CAG AAG TAG ACG TC |
| Biotin | | Bt11 HH | TTT TTA AGA AAC CCT TAC TCT AGC GAA GAT |
| labelled | | | CCT CTT TTT T |
| probe | | | |
| Mud F | | Bt11 MudF | AAG AAA CCC TTA CTC TAG CGA AGA TCC T |
| Mud R | | Bt11 MudR | AGG ATC TTC GCT AGA GTA AGG GTT TCT T |
| Bt176 | | | |
| Head | Forward | Cry2 FMH | CCC ATC GAC ATC AGC CTG AGC |
| primer | | PepC-20MH | ATC TCG CTT CCG TGC TTA GC |
| | Reverse | Cry2 RMH | CAG GAA GGC GTC CCA CTG GC |
| | | Cry04 (SMT- | GGT CAG GCT CAG GCT GAT GT |
| | | CT96) MH | |
| Biotin | | Bt Syn HH | TTT TTA TGT CCA CCA GGC CCA GCA CGT TTT T |
| labelled | | | |
| probe | | | |
| Mud F | | Bt176 MudF | |
| | | BtSyn2 | TCC ACC AGG CCC AGC ACG AAG |
| | | BtSyn3 | AGG CCC AGC ACG AAG CCG G |
| | | Bt176-cryA1-1904 | TGA GCA ACC CCG AGG TGG AGG TG |
| MudR | | Bt176 MudR | |
| | | BtSyn2 MudR | CCG GCT TCG TGC TGG GCC TGG TGG A |
| | | Bt176 MudR2504 | CAC CTC CAC CTC GGG GTT GCT CA |
| 35 S | | | |
| Head | Forward | 35SH-1 | GCT CCT ACA AAT GCC ATC A |
| primer | | 35SMH-F2 | GAA GAT AGT GGA AAA GGA AGG TGG C |
| | | 35SMH-F3 | GGA AAC CTC CTC GGA TTC CAT |
| | Reverse | | |
| | | 35SMH-R1 | CCC TTA CGT CAG TGG AGA TAT CAC AT |
| | | 35SMHH-R2 | CTT GCT TTG AAG ACG TGG TTG G |
| | | 35SMH-R3 | GAT GCT CCT CGT GGG TGG G |
| Mud F | | 35S MudF | GAA AGG CCA TCG TTG AAG ATG C |
| | | 35S Mud2F | TGC CGA CAG TGG TCC CAA AGA TGG A |
| Mud R | | 35S MudR | GGC ATC TTC AAC GAT GGC CTT TC |
| | | 35S Mud2R | TCC ATC TTT GGG ACC ACT GTC GGC A |
| Amp | | | |
| Head | Forward | Ampres FMH | TGC TCA CCC AGA AAC GCT G |
| primer | | | |
| | Reverse | Ampres RMH | TTC TTC GGG GCG AAA ACT CTC |
| Mud F | | Amp pro | GTA AAA GAT GCT GAA GAT CAG TTG GGT GCA |
| Mud R | | Amp MudR | TGC ACC CAA CTG ATC TTC AGC ATC TTT TAC |
| Nos | | | |
| Head | Forward | Nos FMH | GAA TCC TGT TGC CGG TCT TG |
| primer | | | |
| | Reverse | Nos RMH | AAT TTA TCC TAG TTT GCG CGC TA |
| Mud F | | Nos pro | TTT ATG AGA TGG GTT TTT ATG ATT AGA GTC CCG |
| Mud R | | Nos MudR | CGG GAC TCT AAT CAT AAA AAC CCA TCT CAT AAA |
| IPC | | | |
| Synthetic | | IPC | CGC AGC GTT TCA AGC AGC ACA TCA TCG ATC TAA |
| sequence | | | TCG AGC AGA CGG TAC GAT CAG ACG CTG TCA TAC |
| | | | GCA TAA TCG ATA CGC GAT ACT GCC CGC TAA CTG G |
| Forward | | IPC-F | CGC AGC GTT TCA AGC AGC |
| Reverse | | IPC-R | CCA GTT AGC GGG CAG TAT CG |
| Head | Forward | IPC-FMH | CGC AGC GTT TCA AGC AGC |
| primer | | | |
| | Reverse | IPC-RMH | CCA GTT AGC GGG CAG TAT CG |
| Mud F | | IPC pro | AGC AGA CGG TAC GAT CAG ACG CTG T |
| Mud R | | IPC MudR | ACA GCG TCT GAT CGT ACC GTC TGC T |
| Maize | | | |
| Ref gene | | | |
| Head | Forward | ZM1 FMH | TTG GAC TAG AAA TCT CGT GCT GA |
| primer | | | |
| | Reverse | ZM1 RMH | GCT ACA TAG GGA CCC TTG TCC T |
| Biotin | | ZM1 HE | TTT TTC AAT CCA CAC AAA CGC ACG CGT ATT TTT |
| labelled | | | |
| probe | | | |
| Mud F | | MudF | CAA TCC ACA CAA ACG CAC C |
| Mud R | | Mud R | CGT GCG TTT GTG TGG ATT G |

* All Head primers contain the head sequence at the 5′- end in addition to the sequences listed

### Example 1

### Qualitative Multiplex Detection

This example shows that qualitative multiplex detection is possible. The multiplex method was used to detect Bt11 corn (DNA from 2% reference material), Bt176 corn (1%) and Mon810 corn (1%) alone or in combinations (FIG. 3). A corn reference gene detection system was also included to detect corn DNA as such. Each sample was analysed with 2 parallels.

### Example 2

### Quantitative Nature of the PCR Assay

This example shows the quantitative nature of the PCR. Bt11 DNA was diluted with non-GM corn DNA to give different GM concentrations. These were analysed using the multiplex assay (FIG. 4). The signals could be detected directly by fluorescence scanning (not shown) or after enzymatic enhancement (FIG. 4). The gradually fading signals as the concentration of GMO decreases show that the assay is quantitative.

### Example 3

### Eight Plex Detection of GM Maize Constructs

1% Bt176 and 2% Bt11 DNA was detected in an eight-plex reaction (FIG. 5). For Bt176 we obtained signals from the Bt176 construct specific target, the amp target, 35S promoter and maize specific reference gene and finally from the IPC control. The Bt11 sample gave signals with the Bt11 construct specific PCR, the NOS terminator, the 35S promoter and the maize reference gene in addition to the IPC. Weak signals were (and are essentially always) obtained with the amp primers even when no amp resistance genes from GMOs are present. This is probably due to contamination with amp resistance gene from the DNA polymerase preparation.

### Example 4

### Quantitative Nature of the 8-Plex PCR and the effect of Removing the “Head Primers” (Bipartite Primers) After the 1. PCR Step.

This experiment was done to show that it is necessary to remove the “headprimers” after the 1. PCR step to maintain the quantitative nature of the assay. Quantitative 8-plex PCR for detection of GMP corn was carried out. Bt176 DNA and Bt11 DNA were kept constant at 0.7% in all samples. Concentrations of Mon810 DNA was varied from 2.0 to 0%. In FIG. 6A, the PCR was carried out in two steps: 1. PCR (10 cycles) using specific primers with a common “head” sequence. Primers were then digested and the 2. PCR (30 cycles) is carried out using the common head primer. FIG. 6B shows the same as FIG. 6A except that the specific primers were not degraded before the 2.PCR step. FIG. 6A shows clearly the quantitative nature of the assay as the Mon810 DNA signal is gradually fading as the concentration decreases. Even though the Mon810 signals are decreasing in FIG. 6B, it is easily seen that the overall results are dramatically influenced by not removing the “head primers” after the 1 PCR step. The relative signal strength from the different PCRs is changed and the signals are generally weaker. This is most probably caused by different amplification efficiencies of the specific primers with the headsequence and formation of primer dimers.

### Example 5

### Effect of Omitting the 2. PCR Step.

This example showed the effect of omitting the 2.PCR step (FIG. 7). The experiment was the same as in Example 4 (FIG. 6A) except that the 1. PCR step using specific primers with head sequence was extended to 40 cycles and the 2. PCR step was omitted. As in example 4, FIG. 6B performing the PCR with the headprimers present leads to different amplification efficiencies for the different PCRs and some fragments (e.g. Bt176 and Bt11) were not amplified.

### Example 6

### The Effect of Diluting the Template DNA

A reference mixture of 0.7% Bt176, 0.7% Bt11 and 0.7% Mon810 at different dilutions was used as templates in the PCR (FIG. 8). We see that the signals gradually fade as the template DNA is diluted, but that the dilution effect is relatively small down to 16 fold dilution.

### Example 7

### Quantitative Detection of Mon810 Alone and Together with Bt11

A dilution series containing different amounts of Mon810 DNA was analysed alone and in combination with 1% Bt11 in the samples. The fluorescence signals after hybridisation of the labelled probes and the blot after HRP colouring are shown in FIG. 9. The fading of the Mon810 signals as the amount of Mon810 DNA is lowered is clearly visible. The 35S signal decreases in A down to zero as expected and down to a fixed level caused by the presence of Bt11 DNA in B. The other signals remain constant. The fluorescence signals from Mon810 (FIG. 9 panel I) were quantified and plotted against the given concentrations (FIG. 10). A linear response was observed up to 5% Mon810. Little difference was observed between parallels. The signal strengths remained the same whether Bt11 DNA was present or not.

### Example 8

### Quantitative Detection of Mon810 Alone and Together with Bt11 (Repetition)

To investigate the repeatability of the system, the experiment in example 7 was repeated. A dilution series containing different amounts of Mon810 DNA was analysed alone and in combination with 1% Bt11 in the samples. The fluorescence signals and the blot after HRP colouring are shown in FIG. 11. The fading of the Mon810 signals as the amount of Mon810 DNA is lowered is again clearly visible, although the signals have reached some degree of saturation and the difference between signals at higher concentrations of Mon810 is smaller. The 35S signal decreases in A down to zero as expected and down to a fixed level caused by the presence of Bt11 DNA in B. The other signals remain constant. The fluorescence signals from Mon810 were again quantified and plotted against the given concentrations (FIG. 12). An almost linear response was observed up to 2% Mon810. The signal at 5% Mon810 was lower than expected probably due to saturation of the probe (all probe molecules were already labelled). The difference between parallels were greater than at example 7. Again the signal strengths remained the same whether Bt11 DNA was present or not.

### Example 9

### Quantitative Detection of Bt176 Alone and Together with Mon810

The experiment was performed as in example 6, except that the amount of Bt176 was varied and Mon810 was kept constant. A dilution series containing different amounts of Bt176 was analysed alone and in combination with 1% Mon810 in the samples. The flueorescence signals and the blot after HRP colouring are shown in FIG. 13. The fading of the Bt176 signals as the amount of Bt176 DNA is lowered is clearly visible. The 35S signal and the amp signal decrease in A down to zero as expected. 35S decreases down to a fixed level caused by the presence of Mon810 DNA in B. The other signals remain constant. The fluorescence signals from Mon810 were again quantified and plotted against the given concentrations (FIG. 14). Also here a (close to) linear response was observed.

Table 2 below gives details of primers and probes for use in Examples 10, 11 and 12 as well as preferred oligonucleotides for use in the earlier Examples. In this table there are no biotin labelled probes and as shown in Table 1, it is understood that a probe complementary to e.g. Bt11 MudF may also be used in the labelling and capture step.

TABLE 2
Primers, probes and template DNA used in multiplex-PCR and 5′-nuclease PCR.

| | | | |
|---|---|---|---|
| Template | Orientation | Name | Sequence (5′->3′) |

Primers and probes for the multiplex system

| | | | |
|---|---|---|---|
| HEAD | | H | TGC TAT GCG CGA GCT GCG |
| sequence | | | |
| Mon810¹ | Sense^{§} | Mon810F1101MH | H-AAT AAA GTG ACA GAT AGC TGG GCA |
| | Antisense^{§} | Mon810R1101MH | H-CCT TCA TAA CCT TCG CCC G |
| | Probe^{**} | Mon810 Mud1101 | ACG AAG GAC TCT AAC GTT TAA CAT CCT TTG C |
| Bt11 | Sense | AHJ-2MH | H-CGC ACA ATC CCA CTA TCC TT |
| | Antisense | Bt11 RBMH | H-GCC TCC CAG AAG TAG ACG TC |
| | Probe | Bt11 MudF | AAG AAA CCC TTA CTC TAG CGA AGA TCC T |
| Bt176 | Sense | PepC-20MH | H-ATC TCG CTT CCG TGC TTA GC |
| | Antisense | Cry04(SMT-CT96)MH | H-GGT CAG GCT CAG GCT GAT GT |
| | Probe | Bt176-cryA1-1904 | TGA GCA ACC CCG AGG TGG AGG TG |
| T25² | Sense | T25 FMHB | H-CCA GTT AGG CCA GTT ACC CAG A |
| | Antisense | T25 RMHB | H-TGG GAA CTA CTC ACA CAT TAT TAT AGA GAG |
| | Probe | T25 Mud | AGA CTG GTG ATT TCA GCG GGC ATG |
| GA21² | Sense | GA21 FMHB | H-AGC CTC GGC AAC GTC AGC |
| | Antisense | GA21 RMHB | H-TCT CCT TGA TGG GCT GCA G |
| | Probe | GA21 MUDR | AAG GAT CCG GTG CAT GGC CGG |
| | Probe capture | GA21 MUD | GCC GGC CAT GCA CCG GAT CCT T |
| DBT418 | Sense | DBT418 FMHB | H-GTC ATT TCA GGA CCA GGA TTC AC |
| | Antisense | DBT418 RMHB | H-CCT CTA TTC TGG ATG TTG TTG CC |
| | Probe | DBT418 MUDR | GAA GAA TTC AGC CTA ACC AAG TCG CCT C |
| CBH351 | Sense | CBH351 FMHB | H-GGT CAG ATC GTG AGC TTC TAC CA |
| | Antisense | CBH351 RMHB | H-CGC ATG AAA GCT TCC CAG AT |
| | Probe | CBH351 MUD | GCT GAA CAC CCT GTG GCC AGT GAA |
| 35S | Sense | 35SH-1 | H-GCT CCT ACA AAT GCC ATC A |
| | Antisense | 35SMHH-R2 | H-CTT GCT TTG AAG ACG TGG TTG G |
| | Probe | 35S Mud2F | TGC CGA CAG TGG TCC CAA AGA TGG A |
| Amp | Sense | Ampres FMH | H-TGC TCA CCC AGA AAC GCT G |
| | Antisense | Ampres RMH | H-TTC TTC GGG GCG AAA ACT CTC |
| | Probe | Amp pro | GTA AAA GAT GCT GAA GAT CAG TTG GGT GCA |
| Nos | Sense | Nos FMH | H-GAA TCC TGT TGC CGG TCT TG |
| | Antisense | Nos RMH | H-AAT TTA TCC TAG TTT GCG CGC TA |
| | Probe | Nos pro | TTT ATG AGA TGG GTT TTT ATG ATT AGA GTC CCG |
| IPC | Forward | IPC-FMH | H-CGC AGC GTT TCA AGC AGC |
| | Reverse | IPC-RMH | H-CCA GTT AGC GGG CAG TAT CG |
| | Probe | IPC pro | AGC AGA CGG TAC GAT CAG ACG CTG T |
| ZM ref | Sense | ZM1 FMH | H-TTG GAC TAG AAA TCT CGT GCT GA |
| | Antisense | ZM1 RMH | H-GCT ACA TAG GGA GCC TTG TCC T |
| | Probe | MudF | CAA TCC ACA CAA ACG CAC G |

Primers and DNA used for template construction

| | | | |
|---|---|---|---|
| T25 | | T25 1-5′ | GCC AGT TAG GCC AGT TAC CCA |
| | | T25 1-3′ | TGA GCG AAA CCC TAT AAG AAC CCT |
| GA21 | | GA21 F | AGC CTC GGC AAC GTC AGC |
| | | GA21 R | TGT CCT TGA TGG GCT GCA G |
| DBT418 | Template | DBT418hele | GTC ATT TCA GGA CCA GGA TTC ACT GGA GGC GAC |
| | | | TTG GTT AGG CTG AAT TCT TCC GGC AAC AAC ATC |
| | | | CAG AAT AGA GG |
| CBH351 | Template | CBH351hele | GGT CAG ATC GTG AGC TTC TAC CAG TTC CTG CTG |
| | | | AAC ACC CTG TGG CCA GTG AAC GAC ACC GCC ATC |
| | | | TGG GAA GCT TTC ATG CG |
| 35S | Sense | P35S 1-5′ | ATT GAT GTG ATA TCT CCA CTG ACG T |
| | Antisense | T35S 1-3′ | ACT AAG GGT TTC TTA TAT GCT CAA CA |
| IPC | Forward | IPC-F | CGC AGC GTT TCA AGC AGC |
| | Reverse | IPC-R | CCA GTT AGC GGG CAG TAT CG |
| | Template | IPC-T | CGC AGC GTT TCA AGC AGC ACA TCA TCG ATC TAA |
| | | | TCG AGC AGA CGG TAC GAT CAG ACG CTG TCA TAC |
| | | | GCA TAA TCG ATA CGC GAT ACT GCC CGC TAA CTG |
| | | | G |

5′-nuclease PCR primers and probes^{§§}

| | | | |
|---|---|---|---|
| Bt11 | Sense | Fbt11-enhpatjun-AHJ-1 | CTT GGC GGC TTA TCT GTC TC |
| | Antisense | Rbt11-enhpatjun-AHJ-2 | GCT GCT GTA GCT GGC CTA AT |
| | Probe | Fam-Bt11-enh-pat^{*} | TCG ACA TGT CTC CGG AGA GGA GAC C |
| Bt176 | Probe | Bt176-CryA1t | CTG AGC AAC CCC GAG GTG GAG GT |
| T25 | Sense | T25 1-5′ | GCC AGT TAG GCC AGT TAC CCA |
| | Antisense | T25 1-3′ | TGA GCG AAA CCC TAT AAG AAC CCT |
| | Probe | T25 pro^{*} | GCA TGC CCG CTG AAA TCA CCA GTC T |
| DBT418 | Sense | DBT418 F | GTC ATT TCA GGA CCA GGA TTC AC |
| | Antisense | DBT418 R | CCT CTA TTC TGG ATG TTG TTG CG |
| | Probe | DBT418 pro^{*} | GGA GGC GAC TTG GTT AGG CTG AAT TCT TC |
| CBH351 | Probe | CBH351 pro^{*} | TGC TGA ACA CCC TGT GGC CAG TGA |
| Zein³ | Sense | Zetm1 | TGT TAG GCG TCA TGA TCT GTG G |
| | Antisense | Zetm3 | TGC AGC AAC TGT TGG CCT TAC |
| | Probe | Zetmp^{*} | ATC ATC ACT GGC ATC GTC TGA AGC GG |

^{§}All sense and antisense primers used in the MQDA-PCR contain the HEAD sequence, designated by an H at the 5′-end in addition to the given sequence.
^{*}All 5′-nuclease PCR probes contain 5′FAM (6-FAM) and 3′Tamra.
^{§§}All filter bound capture probes are complementary to their corresponding probes
## Only primers and probes different from those used in the MQDA-PCR are listed. All 5′-nuclease primers are without the HEAD sequence.
¹Holck, A., M. Va+E,umla itilingom, L. Didierjean, and K. Rudi. 2002. 5′-nuclease PCR for quantitative event-specific detection of the genetically modified Mon810 MaisGard maize. European Food Research and Technology 214: 449-453.
²Matsuoka, T., Kuribara, H., Akiyama, H., Miura, H., Goda, Y., Kusakabe, Y., Isshiki, K., Toyoda, M., Hino,A. 2001. A multiplex PCR method of detecting recombinant DNAs from five lines of genetically modified maize. Journal of the Food Hygienic Society of Japan 42: 24-32.
³Vaïtilingom, M., Pijnenburg, H., Gendre, F., Brignon, P. 1999. Real-Time Quantitative PCR detection of genetically modified Maximizer Maize and Roundup Ready Soybean in some representative foods. Journal of agricultural and food chemistry 47:5261-5266

### Example 10

### Twelve-Plex PCR to Detect Seven Different GM Maize

The multiplex system was expanded from an eight-plex PCR to a twelve-plex PCR through the inclusion of primers for detection of the maize constructs CBH351, DBT418, GA21 and T25 (FIG. 15). Mixtures containing 0.7 or 1.0% of each of all seven different GM constructs were amplified in one reaction together with the amplicons from amp, nos, 35S, IPC and the maize reference genes. When CBH351, DBT418, GA21 and T25 were amplified separately, a dose response was observed (FIG. 16). No cross reactions during hybridisation were detected. The specific signals from the mixtures of the seven GM constructs were generally weaker than those obtained with pure samples (FIG. 16). This may come from a slightly higher frequency of side reactions when multiple targets are amplified simultaneously. In some cases the standard curves deviate from linearity. This is particularly observed when signals are strong and stems from saturation of targets during the probe labeling reaction. This may be adjusted by lowering the number of labeling cycles. Since reference materials are used in each experiment quantifications are nevertheless possible.

### Example 11

### Analysis of 17 Food and Feed Samples from USA

Seventeen different food and feed samples were screened using the twelve-plex system described in Example 10 (FIGS. 15 and 16). Ten samples were GM positive, and seven GM negative (GM content <0.1%). Eight GM positive samples contained Mon 810, eight contained Bt11 and seven contained Bt176. One sample contained appreciable amounts of GA21, a few more samples harboured small mounts of GA21 and in one sample small amounts of T25 was observed. No maize material containing the constructs CBH 351 or DBT418 was detected. These results are reasonable since the latter four GM maizes are either withdrawn from the market or known to be not very widespread. All the GM negative samples also tested negative when analysed for Bt11, Bt176 and Mon810 by 5′-nuclease PCR. Two GM positive samples contained only marginal amounts of DNA as judged by the increase in intensity of the IPC signal and the Ct values of the 5′ nuclease PCR. Quantification of GM content in these samples was therefore not possible (Ct for GM constructs >40).

### Example 12

### Comparison of Quantification Between Eightplex-PCR and 5′ Nuclease PCR (Taqman PCR)

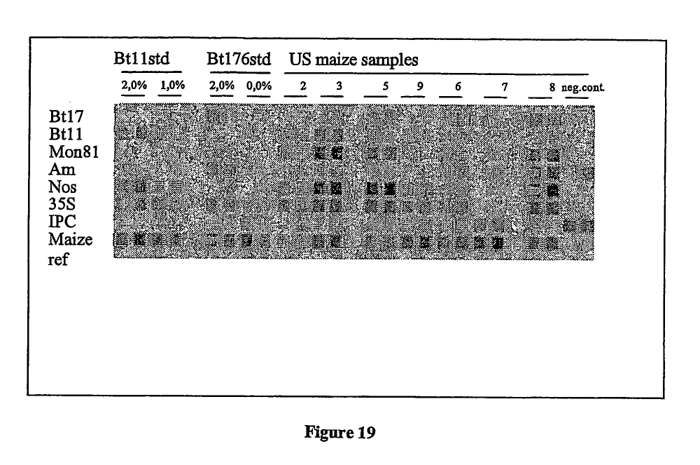

Seven GM positive samples and one negative sample were selected for a comparative quantitative study between the eight-plex PCR and 5′-nuclease PCR for the constructs Mon810, Bt11 and Bt176 (FIGS. 18 and 19). The samples were analysed together with known reference samples. The results of the quantifications are summarized in Table 3 below. Both the eightplex-PCR and 5′ nuclease PCR require known standards for quantification. That is, accurate quantifications can only be obtained if reference materials are analysed in the same experiment. The quantitative results obtained using the calibration curves based on the reference material gave good agreement between the eightplex-PCR and the 5′-nuclease approach. The multiplex method accurately identified samples with high and low content of GM material. In 20 out of 23 analyses the samples could be quantified as containing more than 2%, between 1 and 2%, between 1% and 0.1% or less than 0.1% by both methods. If all the other GM negative samples (<0.1% GM material) are included the corresponding figures are 43 out of 47, respectively. The average standard deviations for the multiplex PCR is comparable to those of the 5′ nuclease PCRs (FIG. 20). When pooled, the pooled average standard deviation was statistically not different from that of the pooled standard deviation of 5′ nuclease PCR. The pooled average standard deviation of the multiplex PCR was also not different from the pooled standard deviation of a 5′ nuclease PCR ringtrial encompassing six maize meal samples analysed by nine laboratories in Europe.

TABLE 3
GM quantification comparisons between eight-PCR and 5′-nuclease PCR on food and feed samples

| | | Mon 810 | | Bt 11 | | Bt 176 | |
|---|---|---|---|---|---|---|---|
| Sample # | Origin | 8plex-PCR | 5′ nuclease | 8plex-PCR | 5′ nuclease | 8plex-PCR | 5′ nuclease |
| 1 | Maize grain^{$} | 1.1 ± 0.2 | 1.1 ± 0.3 | <0.1 | <0.1 | Nd* | 18.6 ± 9.1 |
| 2 | Dog food | >2 | 4.4 ± 3.5 | >2 | 11.5 ± 5.2 | 0.8 ± 0.2 | 0.6 ± 0.1 |
| 3 | Dog food | >2 | 8.5 ± 2.4 | >2 | 5.2 ± 0.0 | 0.8 ± 0.3 | 0.5 ± 0.1 |
| 5 | Chicken feed | >2 | 35.1 ± 23 | 0.6 ± 0.4 | 2.0 ± 0.9 | 0.7 ± 0.0 | 2.1 ± 0.2 |
| 9 | Instant corn mix | <0.1 | <0.1 | 0.8 ± 0.5 | 0.2 ± 0.1 | <0.1 | <0.1 |
| 6 | Corn meal | 0.3 ± 0.0 | 0.2 ± 0.2 | 0.2 ± 0.3 | 0.1 ± 0.0 | 1.6 ± 0.8 | 1.5 ± 0.3 |
| 7** | Whole kernel corn | <0.1 | <0.1 | <0.1 | <0.1 | <0.1 | <0.1 |
| 8 | Yellow corn meal | >2 | 13.6 ± 2.0 | 1.4 | 0.6 ± 0.7 | >2 | 12.9 ± 2.7 |

^{$}horse feed
*nd not determined
**A negative sample included

### Example 13

Use of Uracil DNA Glycosylase (UNG) to Degrade modified bipartite primers

Bipartite primers were designed to amplify the glnA gene from Campylobacter jejuni and constructed to contain uracil instead of thymine at all (appropriate) positions. The “head” primers consisted of part B only and did not contain any uracil.

| | |
|---|---|
| Forward; | 5′GCAGGCUGCUCAUGUCUG |
| | UAGGAACUUGGCAUCAUAUUACC3′ |
| Reverse; | 5′GCAGGCUGCUCAUGUCUG |
| | UUGGACGAGCUUCUACUGGC3′ |
| Head; | 5′GCAGGCTGCTCATGTCTG3′ |

Both sets of primers were added at the start of the PCR reaction. A first amplification reaction was performed and after 10 amplification cycles UNG was added to degrade the bipartite primers. After this degradation step, amplification of the target sequence was still observed.

### Example 14

### Use of Uracil DNA Glycosylase (UNG) to Generate “Head Primers” from the Bipartite Primers

Bipartite primers were designed to amplify the glnA gene from Campylobacter jejuni. Part A of these primers was specific for the gluA sequence and contained uracil instead of thymine, whereas part B did not contain any uracil.

| | |
|---|---|
| Forward; | 5′GCAGGCTGCTCATGTCTG |
| | UAGGAACUUGGCAUCAUAUUACC3′ |
| Reverse; | 5′GCAGGCTGCTCATGTCTG |
| | UUGGACGAGCUUCUACUGGC3′ |

At the start of the PCR reaction, the bipartite primers were the only primers present. After 5 amplification cycles UNG was added to degrade part A of the bipartite primers. This degradation was detected by gel electrophoretic analysis. This reaction thus generated primers which only contained part B, and could participate in a second amplification reaction. Amplification was indeed observed after the UNG degradation step.

1. A method of simultaneously amplifying a plurality of target sequences within sample nucleic acid which comprises:
(a) contacting said sample nucleic acid with one or more primer pairs under conditions which allow hybridisation of the primers to the sample nucleic acid, each primer having a bipartite structure A-B wherein part A is specific for a particular target sequence within the sample nucleic acid and part B is a constant sequence which is common to all primers or is common amongst all forward primers with a different sequence common amongst all reverse primers;
(b) performing a first amplification reaction;
(c) degrading the bipartite primers or separating them from the amplification products of the first amplification reaction;
(d) contacting the amplification products from the first amplification reaction with primers which comprise part B of the bipartite primers or a nucleotide sequence which is substantially identical to part B, under conditions which allow hybridisation of the primers to the amplification products; and
(e) performing a second amplification reaction.

2. A method as claimed in claim 1 wherein the constant region B of the bipartite primers is common between both forward and reverse primers.

3. A method as claimed in claim 1 wherein the constant region B is 10-40 nucleotides in length.

4. A method as claimed in claim 1 wherein the first amplification reaction comprises no more than 25 amplification cycles.

5. A method as claimed in claim 1 wherein step (c) comprises contacting the bipartite primers with a DNA-modifying enzyme so as to cause degradation thereof.

6. A method as claimed in claim 5 wherein step (c) comprises contacting the bipartite primers with an exonuclease so as to cause degradation thereof.

7. A method as claimed in claim 5 wherein the bipartite primers contain one or more uracil residues.

8. A method as claimed in claim 7 wherein the modifying enzyme is uracil DNA glycosylase.

9. A method as claimed in claim 7 wherein the bipartite primers contain no thymine residues.

10. A method as claimed in claim 7 wherein the bipartite primers contain uracil in part A but not part B.

11. A method as claimed in claim 1 wherein step (c) comprises isolating the amplification products from the initial reaction mixture.

12. A method as claimed in claim 11 wherein the amplification products of the first amplification reaction are captured on a solid support.

13. A method as claimed in claim 12 wherein the amplification products are contacted with a probe incorporating a binding partner for a binding moiety provided on said solid support.

14. A method as claimed in claim 1 wherein all of steps (a)-(e) are performed in one reaction vessel.

15. A method as claimed in claim 1 wherein 4 or more target sequences are amplified simultaneously.

16. A method as claimed in claim 1 wherein one or more of the target sequence comprises a non-naturally occurring nucleotide sequence.

17. A method as claimed in claim 16 wherein the target sequence comprises regions which are not naturally found in juxtaposition.

18. A method as claimed in claim 1 wherein one or more of the primer pairs is designed to hybridise either side of a junction region between a regulatory region and a coding region within sample nucleic acid.

19. A method as claimed in claim 1 wherein the sample nucleic acid comprises host organism nucleic acid and a genetically engineered construct.

20. A method as claimed in claim 19 wherein one or more of the target sequences spans a region which comprises both host organism nucleic acid and inserted nucleic acid from the genetically engineered construct.

21. A method as claimed in claim 1 wherein the products of the second amplification reaction are contacted with a plurality of different probes designed to hybridise to the target sequences under conditions which allow hybridisation thereof.

22. A method as claimed in claim 21 wherein the probes which hybridise to the target sequences are labelled at their 3′ end.

23. A method as claimed in claim 22 wherein the labelled probes are captured on a solid support.

24. A method as claimed in claim 1 wherein a known concentration of a control nucleic acid sequence is added to the sample nucleic acid prior to the first amplification reaction.

25. A method as claimed in claim 20 wherein a host species specific sequence is co-amplified with said target sequence which spans a region which comprises both host organism nucleic acid and inserted nucleic acid from the genetically engineered construct.

26. A kit for use in a method of nucleic acid amplification which comprises:
(a) a plurality of bipartite primer pairs of form A-B as defined in claim 1;
(b) means for degrading the bipartite primers or for separating them from the amplification products of a first amplification reaction; and optionally
(c) primers which comprise part B of the bipartite primers of component (a) or a nucleotide sequence which is substantially identical to part B of said primers.
